(19) United States
(12) Patent Application Publication
(10) Pub. No.: US 2005/0089852 A1
(43) Pub. Date: Apr. 28, 2005

(54) Method and compositions for evaluating risk of developing type 2 diabetes in people of chinese descent

(76) Inventors: Shao C. Lee, North Point (CN); Maggie C.Y. Ng, Wanchai (CN); Juliana C.N. Chan, Hong Kong (CN); Julian A.J.H. Critchley, Edinburgh (GB); Christina Parry West, Edinburgh (GB); Clive S Cockram, Hong Kong (CN)

Correspondence Address: Ronald R Santucci;Frommer Lawrence & Haug, 745 Fifth Avenue, New York, NY, 10151, US

(21) Appl. No.: 10/471,446

(22) PCT Filed: Mar. 14, 2002
(86) PCT No.: PCT/CN02/00158

Related U.S. Application Data
(60) Provisional application No. 60/275,891, filed on Mar. 14, 2001.

(51) Int. Cl. C12Q 1/68; C12M 1/34

(52) U.S. Cl. 435/6; 435/287.2

(57) ABSTRACT

Methods and compositions for identifying mutations and polymorphisms in mutant genes encoding gene product involved in insulin secretion, for example, hepatocyte nuclear factor-1∝, glucokinase, amylin and mitochondrial DNA are disclosed. Specifically, a microchip comprising a combination of at least two different mutant genes wherein each gene comprises at least one mutation indicative of a predisposition for type-2 diabetes in a member of a Chinese population is disclosed. A kit comprising the microchip, an isolated nucleic acid, primers and probes which are specifically used to screen or identify the mutations in genes of hepatocyte nuclear factor-1∝, glucokinase, amylin and mitochondrial DNA are also disclosed.

### FIELD OF THE INVENTION

This subject invention relates to the identification and use of mutations and polymorphisms in mutant genes of wild-type genes involved in insulin secretory function that are associated with the increased risk of a Chinese individual to develop type 2 diabetes. The invention is exemplified by a combination of mutations, uniquely identified in Chinese individuals with a positive family history of type 2 diabetes, in the genes encoding hepatocyte nuclear factor-1∝, glucokinase, amylin and mitochondrial DNA. The combination of mutated genes finds use in screening Chinese individuals at risk of developing type 2 diabetes and in providing physicians with information to enable them to apply patient tailored therapies.

### BACKGROUND

Although people of Chinese ancestry account for >20% of the world's population (Chan, et al(1997) 20: 1785), very little is known about the genetic factors that contribute to the development of diabetes in this population. The prevalence of diabetes amongst Chinese people varies from <1% in some rural areas in mainland China to 6-12% in Hong Kong, Singapore, and Taiwan (Chan, et al (1997), supra). Hong Kong can be regarded as a paradigm of future China.

The prevalence of diabetes mellitus is reaching epidemic proportions amongst Hong Kong Chinese, with type 2 diabetes being the predominant form in pateints with early-or late-onset of disease (Chan and Cockram (1997) Diabetes Care 20: 1785). Type 2 diabetes mellitus is a heterogeneous disease that is caused by both genetic and environmental factors. The age-adjusted prevalence of diabetes in the Chinese population has increased from 7.7% in 1990 (Cockram, et al (1993) Diabetes Res and Clin Practice 21: 67) to 8.9% in 1995 (Cockram and Chan (1999) In: Diabetes in the New Millennium, Pot Still Press, Sydney, pp. 11-22). In a population-based study conducted in 1995, the crude prevalence of diabetes mellitus was 9.6%, rising from 1.7% in those aged under 40 years to 25% in those older than 60 years (Janus (1997) Clin Exp Pharmacol Physiol 24: 987). There is a high prevalence of obesity (43%) and positive family history of diabetes (50%) in Chinese patients presenting with acute or early onset diabetes (Chan, et al (1993) Postgrad Med J 69: 204; Ko, et al (1998) 35: 761). These findings indicate that genetic factors, in addition to environmental factors, can be an important cause of early onset diabetes in this population.

Because type 2 diabetes is an insidious disease, it is estimated that as many as half of the individuals in Hong Kong that would be considered diabetic remain undiagnosed. Most patients are finally diagnosed only when presenting with overt symptoms that often are the consequence of advanced disease. Clinic as well as population-based studies reveal that about 17% of diabetic patients in Hong Kong are diagnosed before age 35 years (Chan, et al (1993) Postgrad Med 69: 204; Janus (1996) The Hong Kong cardovascular risk factor prevalence study 1995-1996 Dept of Clin Biochem, Queen Mary Hospital of Hong Kong, Hong Kong, 1997). Due to their anticipated long duration of disease, it is important to classify and characterize the nature of diabetes in these young patients to facilitate early diagnosis and appropriate treatments. Current methods of diagnosing type 2 diabetes generally involve assessing phenotypic parameters, such as measuring fasting serum glucose levels by administering an oral glucose tolerance test (OGTT) to determine impaired glucose tolerance (IGT) or impaired fasting glucose (IFG). Phenotypic assessments of persons suspected of having type 2 diabetes are important, but they are limited in that patients generally receive a diagnosis only after presentation with overt symptoms. Furthermore, because the common symptoms of type 2 diabetes are a consequence of a combination heterogenous genetic and environmental causes, the therapies provided are general with regard to the disease rather than targetted to the specific etiology of the individual patient. Numerous studies have attempted to correlate the increased risk for development of type 2 diabetes with a mutation of a specific gene, but the results of these studies repeatedly demonstrate that no one mutated gene can be attributed as the major cause of type 2 diabetes, emphasizing the heterogeneous nature of this disease. Furthermore, a mutation in a particular gene that correlates with increased risk for developing type 2 diabetes in individuals of one ethnic population is not relevant to individuals of a second ethnic population, wherein the risk for type 2 diabetes in individuals of the second ethnic population will correlate with a different mutation or a mutation in a completely different gene.

It is therefore of interest to identify additional genetic mutations and polymorphisms that are indicative of an increased risk for developing type 2 diabetes in people of Chinese ancestry, and to develop methods that can be effectively employed to prophylactically identify asymptomatic Chinese individuals with a genetic predisposition for type 2 diabetes.

Relevant Literature

Maturity-onset diabetes of the young (MODY) is a monogenic form of diabetes characterized by autosomal dominant inheritance, early onset (usually before 25 years of age) and a primary defect in pancreatic β-cell function (Fajans (1990) Diabetes Care 13: 49; Chan, et al (1990) Diabetic Med 7: 211; Byrne, et al (1996) Diabetes 45: 1503). This form of diabetes can result from mutations in at least five different genes including those encoding the glycolytic enzyme glucokinase (Froguel, et al (1993) New Engl J Med 328: 697), the liver-enriched transcription factors expressed in the pancreatic β-cell, which are hepatocyte nuclear factors HNF-1α (Yamagata, et al (1996) Nature 384: 455), HNF-1β (Horikawa, et al (1997) Nature Genet 17: 384), and HNF-4α (Yamagata, et al (1996) Nature 384: 458), and insulin promoter factor-1 (IPF-1) (Stoffers, et al (1997) Nature Genet 17: 138).

Some mutations and polymorphisms in the glucokinase and HNF-1α genes that are associated with the genetic predisposition of a Chinese individual to develop type 2 diabetes mellitus have been initially identified in Ng, et al (Diabetic Medicine 1999, 16: 956, herein incorporated by reference), but this manuscript does not disclose how these mutations and polymorphisms might be used to identify Chinese individuals with increased risk of developing type 2 diabetes.

U.S. Pat. No. 5,541,060 discloses the results of screening a cohort of sixteen French families having MODY and the identification of several missense mutations in the glucokinase gene, however none of the mutations identified are relevant to individuals of Chinese descent. U.S. Pat. No. 5,800,998 discloses a point mutation at nucleotide 414 of human HNF 1α, but this single point mutation is not associated with a genetic predisposition of a Chinese individual to develop type 2 diabetes.

Major susceptibility loci for non-insulin dependent diabetes have been identified through genome scans of individuals in Mexican-American (Hanis, et al (1996) Nature Genet 13: 161) and Finnish (Mahtani, et al (1996) Nature Genet 14: 90) populations, but not in individuals of a Chinese population. Specific microsatellite regions of genomic DNA can be correlated with major susceptibility loci that closely associate with the increased risk of a Chinese subject to develop type 2 diabetes. For instance, Le Stunff, et al (Nature Genet. (2000) 26: 444) have reported that particular alleles of the insulin gene variable number of tandem repeat (VNTR) locus are associated with obesity and type 2 diabetes. Also, microsatellite polymorphisms flanking the glucokinase have been associated with type 2 diabetes in a Taiwanese population (Wu, et al (1995) Diabetes Res Clin Pract 30: 21).

### SUMMARY OF THE INVENTION

Compositions and methods are provided, wherein a unique combination of genetic markers indicative of a genetic predisposition for developing type 2 diabetes in members of a Chinese population is described. The invention is exemplified by a combination of mutated gene sequences from wild-type genes that are involved in insulin secretory function, including hepatocyte nuclear factor 1α (HNF-1α), glucokinase, amylin and mitochondrial DNA. The combination of representative mutations include G20R, A116V, IVS2nt→GA, R203H, S432C and 1618M of HNF-1α; V101M, 1110T, A119D, Q239R and G385V of glucokinase; S20G of amylin; and A3243G of mitochondrial tRNA^{Leu(UUR)}. The combination of the mutated genes of interest will be most efficiently used for screening individuals at increased risk by attaching them to a microchip.

Embodiments of methods for determining or detecting the genetic predisposition of a Chinese individual to develop type 2 diabetes include obtaining a sample containing genomic nucleic acid from a Chinese patient, such as a tissue biopsy or a blood sample, and contacting that sample with a representative combination of at least two mutated genes of interest, then subjecting the sample DNA together with the patient's DNA to hybridization conditions stringent enough to detect nucleotide differences of at least one base pair. Alternatively, particular genes of interest from the genomic DNA of a Chinese individual at risk are screened using PCR primer pairs and PCR-RFLP techniques to identify the presence or absence of a mutation known to be associated with type 2 diabetes. The methods further encompass screening the genomic DNA of Chinese individuals who have been diagnosed with type 2 diabetes or who have a primary family member with type 2 diabetes for additional associative mutations in identified genes or for mutations correlative with the predisposition of a member of a Chinese population to develop type 2 diabetes in additional candidate genes, such as those associated with diabetic kidney disease and obesity.

The invention further provides for nucleic acid primers and probes that are specifically used to identify mutations, for instance by PCR or hybridization, of wild-type genes involved in insulin secretion that are associated with an increased risk of a Chinese subject to develop type 2 diabetes. Additionally, proteins translated from genes carrying at least one mutation associated with increased risk of a Chinese individual to develop type 2 diabetes find use in functional diagnostic assays and in the production of diagnostic antibodies that bind to the mutant but not the wild-type protein.

The prophylactic detection of mutations and polymorphisms that are indicative of a genetic predisposition of a Chinese individual to develop type 2 diabetes finds application in providing clinicians with information that allows for early detection and therapy initiation before the onset of overt symptoms or complications, and that enables clinicians to administer specifically targetted therapies that address the etiology of an individual's disease.

### BRIEF DESCRIPTION OF DRAWINGS

FIG. 1A shows the nucleic acid sequence of human nuclear factor 1a (HNF-1α) exon 1 with the G20R mutation (SEQ ID NO: 1). The wild-type sequence is GenBank number U72612. FIG. 1B shows the nucleic acid sequence HNF-1α exon 2 with the A116V mutation (SEQ ID NO: 2). The wild-type sequence is GenBank number U72613.

FIG. 2 shows the nucleic acid sequence of HNF-1α exons 3 and 4 depicting the splice acceptor site mutation IVS2nt-1G→A (SEQ ID NO: 3) and the missense mutation R203H (SEQ ID NO: 4). The wild-type sequence is GenBank number U72614.

FIG. 3A shows the nucleic acid sequence of HNF-1α exons 5 and 6 with the S432C mutation (SEQ ID NO : 5). The wild-type sequence is GenBank number U72615. FIG. 3B shows the nucleic acid sequence of HNF-1α exon 10 with the I618M mutation (SEQ ID NO: 6). The wild-type sequence is GenBank number U72618.

FIG. 4A shows the nucleic acid sequence of human glucokinase exon 3, depicting the mutations V101M (SEQ ID NO: 7), I110T (SEQ ID NO: 8) and A119D (SEQ ID NO: 9). The wild-type sequence is GenBank number AF041016. FIG. 4B shows the nucleic acid sequence of human glucokinase exon 7 with the Q239R mutation (SEQ ID NO: 10). The wild-type sequence is GenBank AF041019. FIG. 4C shows the nucleic acid sequence of human glucokinase exon 9 with the G385V mutation (SEQ ID NO: 11). The wild-type sequence is GenBank number AF041021.

FIG. 5 shows the nucleic acid sequence of the human amylin gene exon 3 with the S20G mutation (SEQ ID NO: 12). The wild-type sequence is GenBank number X52819.

FIG. 6 shows the nucleic acid sequence base pairs 3001-3480 of the human mitochondrion complete genome, depicting the A3243G mutation (SEQ ID NO: 13). The wild-type sequence is GenBank number J01415.

FIG. 7 shows the pedigrees of families with mutations/polymorphisms in the glucokinase (HK84) or HNF-1α gene (HK10 and HK54). Individuals with diabetes are noted by filled symbols; individuals with impaired fasting glucose (IFG) or impaired glucose tolerance (IGT) by grey symbols; non-diabetic individuals by open symbols and untested by hatched symbols. The arrow indicates the proband. Present age, age at diagnosis and genotype of glucokinase or HNF-1α of tested individuals are noted: N, normal; M, mutation/polymorphism.

FIG. 8 shows the pedigrees of families with an mt3243 mutation. Individuals with diabetes are noted by filled symbols, IGT by grey symbols, non-diabetic individuals by open symbols, and untested individuals by hatched symbols. The arrow indicates the proband. Present age, age of diagnosis, audiogram and genotype are also shown. N, normal; M, mutant allele.

FIG. 9A-9J show the pedigrees of 10 families carrying the HNF-1α (9A-9B), glucokinase (9C-9E), mt3243 (9F-9H) or amylin S20G (9I-9J) gene mutations/polymorphisms. Subjects with diabetes are noted by black symbols, subjects with IFG or IGT by grey symbols, non-diabetic and untested subjects by open symbols. The genotype of the family members is indicated by: N, wild-type allele; and M, mutant/variant allele. Present age, age at diagnosis, therapy and complications are stated in this order. The proband is indicated by an arrow. Abbreviations: Oral, oral drugs; Ins, insulin; R, retinopathy; K, albuminuria; U, neuropathy; H, hearing impairment.

FIG. 10 shows the pedigree of a Chinese family with HNF-1α IVS2nt-1G→A mutation. Subjects with diabetes are represented by black symbols, subjects with IGT by grey symbols and untested ones by open symbols. The genotype of family members is indicated: N, normal allele; and M, mutant allele. The proband is indicated by an arrow. CP, C-peptide; GST, glucagon stimulation test; Complications: R, retinopathy; K, nephropathy; U, neuropathy.

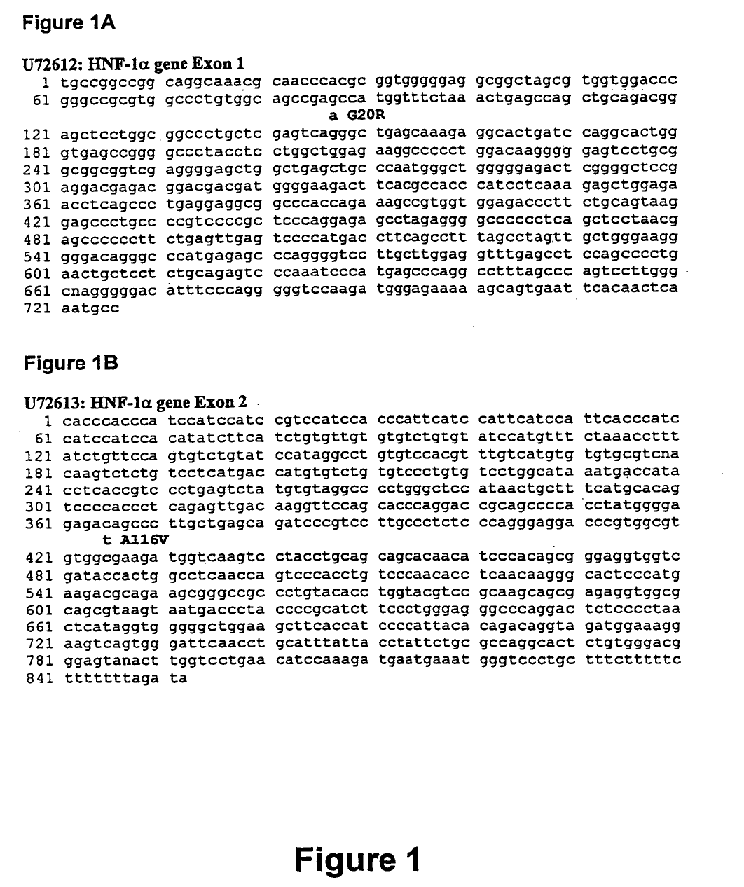

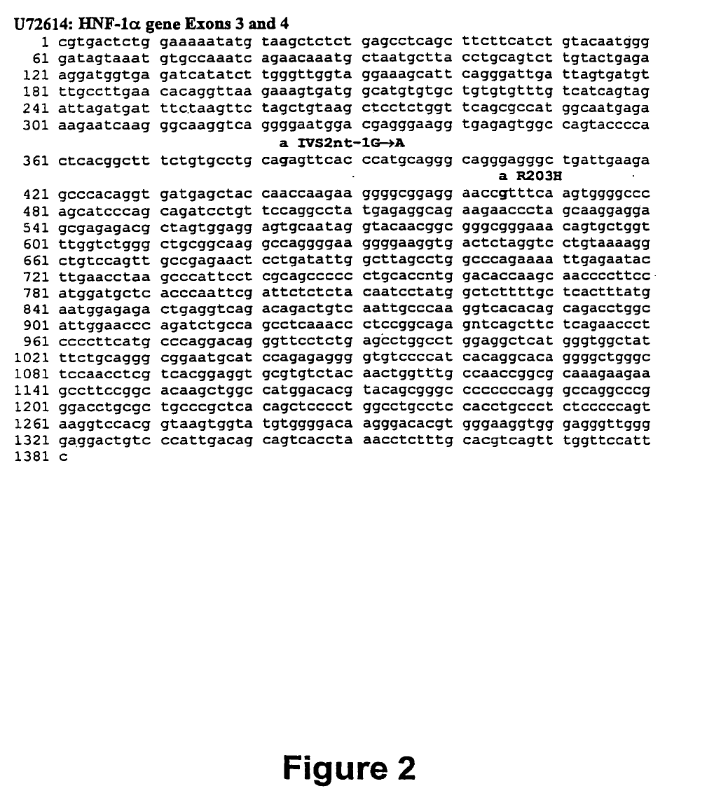

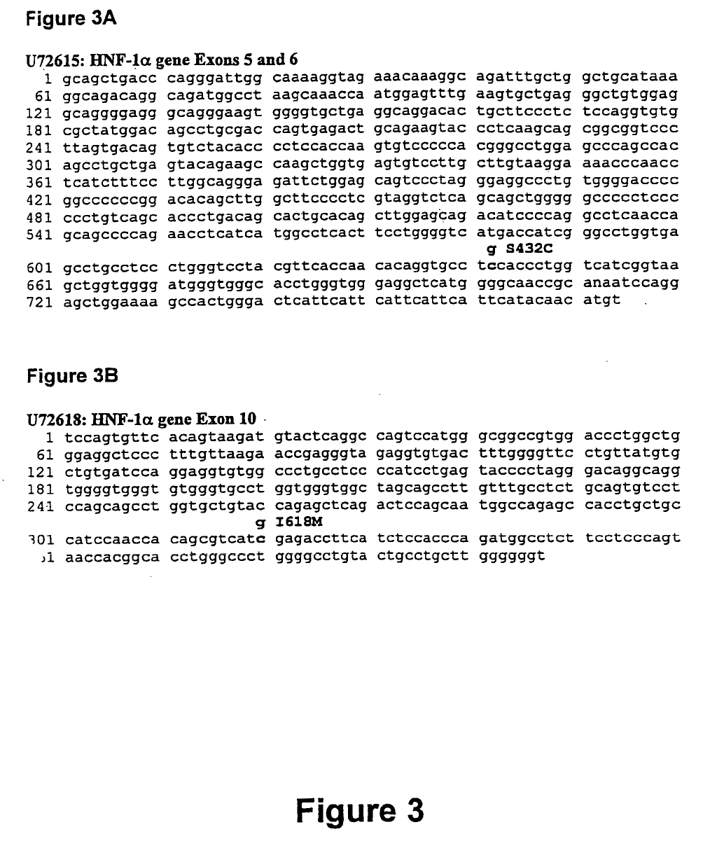

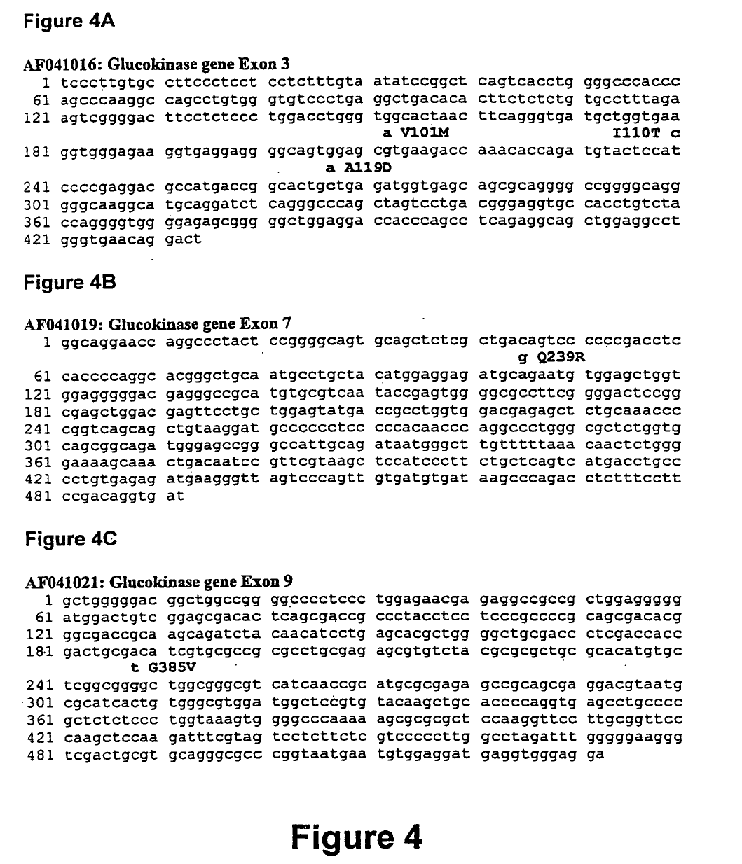

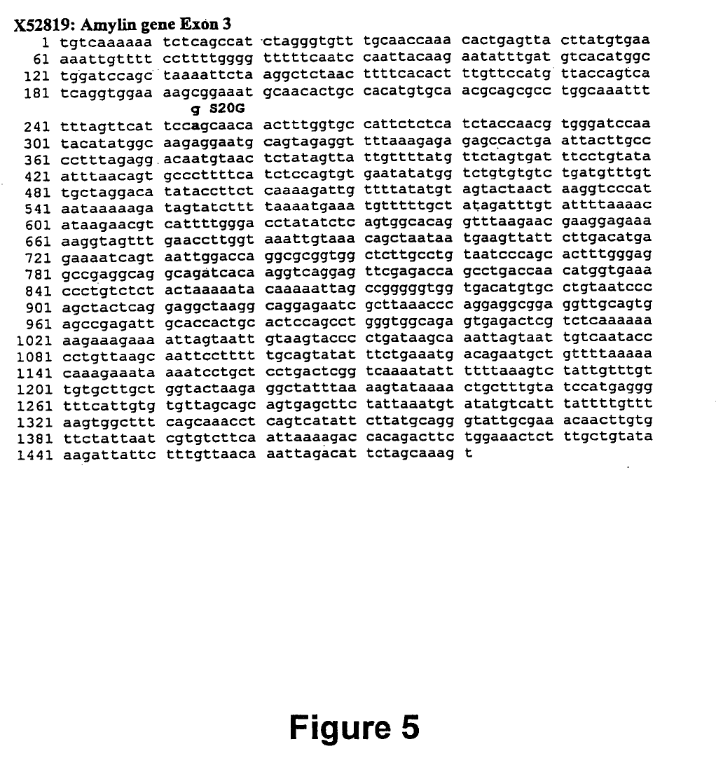

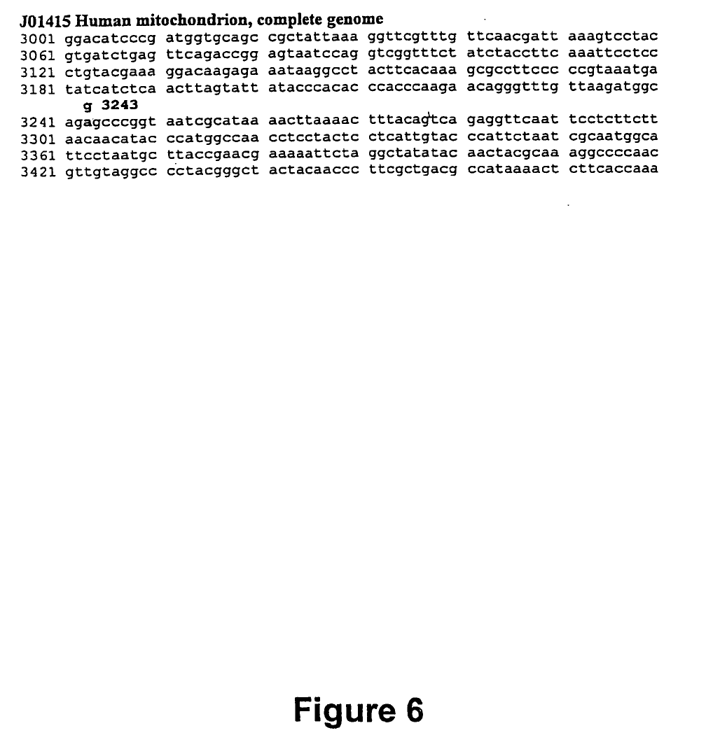

### DESCRIPTION OF THE SPECIFIC EMBODIMENTS

Compositions and methods are provided, wherein a unique combination of genetic markers indicative of a genetic predisposition for developing type 2 diabetes in members of a Chinese population is described. The invention comprises as compositions: (1) a combination of nucleic acid sequences from wild-type genes that encode proteins important for insulin secretory function, each nucleic acid sequence having a mutation uniquely associated with the genetic predisposition of a Chinese individual to develop type 2 diabetes, (2) nucleic acid sequences encoding glucokinase and HNF-1α and carrying previously unreported mutations indicative of the increased risk of a Chinese person to develop type 2 diabetes, (3) a microchip having attached to it at least two of the mutated genes of interest, and (4) nucleic acid primers used to detect the unique mutations in the genes of interest. The methods involve: (1) obtaining genomic DNA from a Chinese subject, (2) combining the genomic DNA with either a combination of the mutated nucleic acids of interest or a combination of primers used to identify the presence or absence of a mutation in a gene of interest, and (3) detecting for the presence or absence of mutations, either by identifying mismatches between the patient's DNA and a wild-type or mutant nucleic acid sequence by hybridization techniques, or by amplifying regions of the patients DNA that contain putative mutations by PCR, and subjecting the amplicons to restriction endonucleases and/or DNA sequencing.

Advantages of the present invention include that the method of screening uses genetic markers shown to cosegregate with type 2 diabetes in persons of Chinese ancestry to assess whether a given patient is at increased risk for developing type 2 diabetes. The mutations and polymorphisms used for screening are specifically applicable to individuals of Chinese descent. As a further advantage, the screening can be based upon the presence or absence of a combination of at least two different mutations or polymorphisms to provide for even more accurate and reliable evaluations because the contributing factors to development of type 2 diabetes are heterogeneous. Identification of particular mutations or polymorphisms in an individual offers the advantage that with this information physicians are able to provide more specific and appropriate therapies for individual patients, and to guide a patient in making lifestyle adjustments to ameliorate or delay symptoms of diabetes and associated complications. Because type 2 diabetes is often an insidious disease, representative combinations of genetic markers indicative of a predisposition in a Chinese individual to develop the disease can be used to screen populations of individuals who may be at increased risk for developing type 2 diabetes so that they can be given appropriate therapy before overt diabetic symptoms or complications are realized. Likewise, family members of an individual diagnosed with type 2 diabetes can be screened for the particular mutants/polymorphisms of the affected individual to quickly identify family members also at increased risk of developing type 2 diabetes.

By a member of a Chinese population is intended to include any individual of Chinese ancestry. In certain cases, for instance when a mutation in a gene involved in the secretion of insulin is dominant for increasing the risk of a Chinese individual to develop type 2 diabetes, a member of a Chinese population will encompass those individuals with at least one parent of Chinese descent. A member of a Chinese population may be more specifically identified by HLA haplotyping. For example, HLA class I and class II frequencies among a Hong Kong Chinese population have been studied by Chang and Hawkins (Hum Immunol (1997) 56: 125). Numerous studies have been carried out to determine HLA class I and class II alleles that are more frequently or even uniquely found in members of a Chinese population, and alleles with strong associations. Shaw et al and Shen et al have studied HLA polymorphism and allele frequency and association of Chinese populations in Taiwan (Tissue Antigens (1997) 50: 610; Tissue Antigens (1999) 53: 51; J Formos Med Assoc (1999) 98: 11). Allele frequency and associations found in Chinese individuals of mainland China have been reported by Trejaut et al (Eur J Immunogenet (1996) 23: 437), Shieh, et al (Transfusion (1996) 36: 818), Zhao et al (Eur J Immunogenet (1993) 20: 293), Wang, et al (Tissue Antigens (1993) 41: 223; Hum Immunol (1992) 33: 129), Lee, et al (Eur J. Immunogenet (1999) 26: 275), and Gao et al (Hum Immunol (1991) 32: 269; Tissue Antigens (1991) 38: 24; Immunogenetics (1991) 34: 401). Additionally, a Chinese individual may be objectively defined by “DNA fingerprinting” techniques well known to those in the art, where microsatellite, short tandem repeat (STR) and variable number tandem repeat (VNTR) loci specific to individuals of Chinese descent are identified. Numerous examples of such ethnic genotyping studies have been reported (Meng, et al (1999) J Forensic Sci 44: 1273; Yoshimoto, et al (1999) Int J Legal Med 113: 15; Wu, et al (1999) J Forensic Sci 44: 1039; Evett, et al (1996) Am J Hum Genet 58: 398; Gill and Evett (1995) Genetica 96: 69; Balazs (1993) EXS 67: 193; Lan, et al (1992) Arch Kriminol 189: 169; and Hwu, et al (1992) J Formos Med Assoc 91: 839). All of these above references are incorporated herein by reference.

Whereas insulin resistance is a strong predictor of type 2 diabetes, it is not sufficient for manifestation of the disease (So, et al (2000) Hong Kong J. Med 6: 69-76). A relative insulin deficiency is essential to the development of hyperglycemia, setting up a vicious cycle wherein elevated glucose levels are toxic to pancreatic β-cells, thereby inducing insulin resistance and decreased β-cell secretory function. Based in the intrinsic interconnection between insulin secretion and action, the invention is exemplified by a combination of mutated gene sequences from wild-type genes that are involved in insulin secretory function, including hepatocyte nuclear factor 1α (HNF-1α), glucokinase, amylin and mitochondrial DNA. By “genes involved in insulin secretory function” and “genes involved in insulin secretion” is intended genes in which a heterozygous mutation has a dominant-negative effect on normal pancreatic β-cell secretory function. The invention is primarily concerned with a representative array of gene markers, the combination of which is uniquely indicative of the genetic predisposition of a member of a Chinese population to develop type 2 diabetes (referred to hereinafter as “genes of interest”). The combination of representative mutations is exemplified by G20R, A116V, IVS2nt→GA, R203H, S432C and 1618M of HNF-1α; V101M, I110T, A119D, Q239R and G385V of glucokinase; S20G of amylin; and A3243G of mitochondrial tRNA^{Leu(UUR)}”. The mutation IVS2nt→GA represents a splice acceptor site mutation that likely results in a truncated translation product.

A representative combination of the mutated genes of interest finds particular use in the prophylactic screening of (i) Chinese individuals who have been diagnosed with maturity onset diabetes of the young (MODY) to determine the etiology of their disease, (ii) Chinese individuals that have a positive family history of type 2 diabetes to determine their likelihood of developing diabetic symptoms, and (iii) and Chinese individuals deemed to be at greater risk of developing diabetic symptoms because of correlative phenotypic characteristics (i. e. obese individuals).

The combination of the mutated genes of interest will be most efficiently used for screening-individuals at increased risk by attaching them to a microchip or other solid support. A specific kind of microchip is not critical, except that it must be able to present a representative array of at least two different nucleic acid sequences, each with a mutation or polymorphism indicative of the increased risk of a Chinese individual to develop type 2 diabetes. Additionally, it will be useful to attach representative wild-type nucleic acid sequences to the chip as comparative controls. The microarray will normally involve a plurality of different nucleic acid sequences, usually be at least 10, more usually at least 20, frequently at least 50, but may have as many as 100 or more. Chips that will find use with the present invention are known in the art (for example, see U.S. Pat. Nos. 5,741,644, 5,837,832 and 6,183,970). Additionally, other solid substrates may be used for the covalent attachment of representative combinations of mutated nucleic acid sequences of interest, including beads and slides. Solid supports can be made out of glass or silicon oxide or other materials that can be adapted to be covalently attached to oligonucleotide sequences by the introduction of functionalities which react with oligonucleotides.

One may use a variety of approaches to bind the nucleic acid to the solid substrate. By using chemically reactive solid substrates, one may provide for a chemically reactive group to be present on the nucleic acid, which will react with the chemically active solid substrate surface. For example, by using silicate esters, halides or other reactive silicon species on the surface, the nucleic acid may be modified to react with the silicon moiety. One may form silicon esters for covalent bonding of the nucleic acid to the surface. Instead of silicon functionalities, one may use organic addition polymers. e. g. styrene, acrylates and methacrylates, vinyl ethers and esters, and the like, where functionalities are present which can react with a functionality present on the nucleic acid. For example, amino groups, activated halides, carboxyl groups, mercaptan groups, epoxides, and the like, may be provided in accordance with conventional ways. The linkages may be amides, amidines, amines, esters, ethers, thioethers, dithioethers, and the like. Methods for forming these covalent linkages may be found in U.S. Pat. No. 5,565,324 and U.S. Pat. No. 6,156,501.

The invention also contemplates a microassay system and a kit that comprises a solid support having attached to it a representative array of nucleic acid sequences, each with a mutation or polymorphism associated with the genetic disposition of a Chinese individual to develop type 2 diabetes. The microassay system or kit would contain, for example, a microchip or beads to which are attached wild-type and mutant nucleic acid sequences from genes that encode proteins involved in insulin secretory function, preferentially wild-type and mutant sequences from HNF-1α, glucokinase, amylin and mitochondrial DNA. Additionally, mutant and wild-type sequences known to hybridize and known not to hybridize under stringent conditions to those sequences immobilized on the support would be included as positive and negative controls, respectively. A microassay system or kit with nucleic sequences immobilized on a solid support would involve screening by hybridization detection (fluorescent or radioactive signal upon duplex formation). Alternatively, another microassay system or kit would include primer pairs that anneal to nucleic acid sequences encoding proteins involved in insulin secretion. The primer pairs specifically anneal to flanking regions of the genes that putatively contain mutations associated with type 2 diabetes, such that PCR amplification with such primers would reveal the presence or absence of an associative mutation of interes. Such a kit or microassay system would also contain representative mutant and wild-type sequences as controls, and screening would be carried out using PCR and sequencing or through PCR restriction fragment length polymorphism analysis (RFLP) and electrophoresis.

Type 2 diabetes is a heterogeneous disease, and no single mutation or single mutated gene can be fully attributed to the manifestation of its symptoms. Therefore, a combination of at least two different nucleic acid sequences encoding mutations or polymorphisms of closely associated with increased risk of a person of Chinese descent to develop type 2 diabetes is attached to a microchip or is individually screened. By “at least two different nucleic acid sequences” is intended two different nucleic acid sequences from the same wild-type gene having different mutations, or two different mutant nucleic acid sequences from two different wild-type genes. Preferably, at least one of the mutant; sequences A116V of HNF-1α (SEQ ID NO: 2), V101M (SEQ ID NO: 7) or Q239R (SEQ D NO: 10) are attached to the microchip or solid support. By wild-type gene is intended one that is not associated with type 2 diabetes, and this would include any allelic variant of the wild-type gene, at any frequency, and that encodes a protein that functions in its expected manner without inducing pathological symptoms. By mutant gene is intended one whose sequence has been modified by insertions, deletions, or substitutions of at least one nucleic acid base pair, wherein the modification may result in detectable changes in the expression or function of the mutant gene product as compared to the wild-type gene product. In the genes of interest for the invention, a mutant gene is associated with type 2 diabetes. The nucleic acid sequences may be from genomic DNA, complementary DNA (cDNA) or from messenger RNA (mRNA). They may be synthetic or isolated from human bodily tissue or fluid. The mutations preferably occur, but do not need to occur, in a translated region of a nucleic acid sequence that encodes a protein that in wild-type form is involved in glucose metabolism or insulin secretion.

Within a translated nucleic acid sequence, a mutation can be a missense mutation, replacing one amino acid with another amino acid, or a nonsense mutation, replacing an amino acid with a stop codon. Mutations can also be insertions or deletions of at least one nucleic acid in either a coding or in non-coding region, such as a region that controls the transcription of a gene, including promoters, enhancers, response elements, signal sequences and polyadenylation signals, and the like. Single nucleotide polymorphisms (SNPs), preferably but not necessarily occurring within the translated regions of nucleic acid sequences that encode proteins involved in glucose metabolism or insulin secretion and that correlate with increased risk of type 2 diabetes are also contemplated by the present invention. Such SNPs can be identified by correlating mutations in known genes that cosegregate with development of type 2 diabetes in members of families with a positive history of the disease. Additionally, SNPs that occur in non-translated and translated regions can be identified through genome-wide scans and correlate linkage analyses of family pedigrees. The use of microarray technologies also can be conveniently applied to identifying SNPs of interest.

Embodiments of methods for determining or detecting the genetic predisposition of a Chinese individual to develop type 2 diabetes include obtaining a sample containing genomic nucleic acid from a Chinese patient, such as tissue from autopsy or biopsy, or a blood sample, and contacting that sample with a representative combination of at least two mutated genes of interest, then subjecting the sample DNA together with the patient's DNA to hybridization conditions stringent enough to detect nucleotide differences of at least one base pair. Alternatively, particular genes of interest from the genomic DNA of a Chinese individual at risk are subjected to restriction fragmentation and then screened using PCR primer pairs and PCR-RFLP techniques to identify the presence or absence of a mutation known to be associated with type 2 diabetes. The methods further encompass screening the genomic DNA of Chinese individuals that have been diagnosed with type 2 diabetes or that have a primary family member with type 2 diabetes for additional associative mutations in identified genes or for mutations correlative with the predisposition of a member of a Chinese population to develop type 2 diabetes in additional candidate genes, such as those associated with diabetic kidney disease and obesity. Mutations are most efficiently identified in Chinese families with a positive history of developing type 2 diabetes (i. e. families with members that develop MODY). However, identified associative mutations are useful for identifying the increased risk in any member of a Chinese population.

In practicing a method of identifying the mutations associated with the genotype of a Chinese individual who is at increased risk for developing type 2 diabetes, Chinese subjects with (i) a confirmed diagnosis of type 2 diabetes, (ii) a positive familial history of type 2 diabetes or (iii) phenotypically determined elevated risk factors (e. g. obesity) are identified by clinical testing, pedigree analysis, and linkage analysis, using standard diagnostic criteria and interview procedures, and DNA or RNA samples are obtained from the subjects.

A sample of genomic DNA is obtained from any nucleated cell source or body fluid. Examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, amniotic fluid, and tissue exudates at the site of infection or inflammation. DNA is extracted from the cell source or body fluid using any of the numerous methods that are standard in the art. It will be understood that the particular method used to extract DNA will depend on the nature of the source.

A variety of techniques are then employed to identify the presence or absence of new or known mutant sequences. First, the sequences of genes known to be involved in insulin secretory function may be subjected to direct DNA sequencing, using methods that are standard in the art. Mutations may be detected using a PCR-RFLP, in which pairs of olignucleotides are used to prime amplification reactions and the sizes of the amplification products, cleaved or uncleaved by restriction endonucleases, are compared with those of control products. Other useful techniques include Single-Strand Conformation Polymorphism analysis (SSCP), denaturing gradient gel electrophoresis, and two dimensional gel electrophoresis, EMC, and the like. Detection of known mutations, such as those exemplified by the invention, may alternatively be detected using nucleic acid probes that contain mutations of interest in sufficiently stringent hybridization conditions.

Appropriate stringency conditions for identifying mutations of at least one base pair in a mutant sequence of a gene involved in insulin secretory function, for example, in 6× sodium chloride/sodium citrate (SSC) at at least 42° C., preferably at about 43,44 or 45° C., followed by a wash of 2×SSC at 50° C., are known to those skilled in the art or can be found in Current Protocols in Molecular Biology, John Wiley & Sons, N. Y. (1989). To optimize conditions, both salt and temperature may be varied, or either the temperature or salt concentration may be held constant while the other variable is changed. For example, the salt concentration in the wash step can be selected from a low stringency of about 2×SSC at 50° C. to a high stringency of about 0.2×SSC at 50° C. The temperature in the wash step can be increased from low stringency conditions at room temperature, about 22° C. to high stringency conditions at about 65° C. Optimal conditions will additionally depend on the length of the nucleic acid probe used, and the scale at which the hybridization takes place. High stringency hybridization conditions using nucleotides attached to a microchip may require lower temperatures. One can perform a series of routine thermal equilibrium experiments to determine optimal hybridization discrimination between wild-type and mutant gene sequences of interest, by starting a low stringency temperature of about 20° C. and increasing the temperature in successive 5° C. temperature intervals.

The nucleic acid probes of the invention are nucleic acid sequences from the mutated genes of interest. They are at least 8,12,15 or 20 base pairs in length, but can be 50,80 or 100 base pairs in length, and may even be 250 or 500 base pairs in length, and include at least one associative mutation but may include multiple mutations, and can be as long as the length of the transcribed gene. The length of the probe chosen will be optimized based on the better base pair mismatch discrimination of shorter probes and the better duplex stability of longer probes (see U.S. Pat. No. 6,156,601 and U.S. Pat. No. 6,197,506, herein incorporated by reference). The length of the probe used should enable discrimination between a mutant and wild-type gene with at least one base-pair mutation.

For detection of hybridized probes, light detectable means are preferred, although other methods of detection may be employed, such as radioactivity, atomic spectrum, and the like. For light detectable means, one may use fluorescence, phosphoresence, absorption, chemiluminescence, or the like. The most convenient will be fluorescence, which may take many forms. One may use individual fluorescers or pairs of fluorescers, particularly where one wishes to have a plurality of emission wavelengths with large Stokes shifts. Illustrative fluorescers which have found use include fluorescein, rhodamine, Texas red, cyanine dyes, phycoerythrins, thiazole orange and blue, etc. When using pairs of dyes, one may have one dye on one molecule and the other dye on another molecule which binds to the first molecule. For example, one may have one dye on the first or bound component and the other dye on the second or complexing component. The important factor is that the two dyes when the two components are bound are close enough for efficient energy transfer (see U.S. Pat. No. 5,992,617).

The identification of the presence or absence of known mutations can also conveniently be detected by PCR followed by restriction analysis and/or sequencing using techniques well known to those in the art. PCR analysis furthermore offers an efficient technique for identifying new mutations in genes already known to contain mutations that correlate with the predisposition of a Chinese individual to develop type 2 diabetes, or in identifying associative mutations in additional candidate genes. PCR primers should be at least 12 base pairs in length, preferably 15-18 base pairs in length, and may be as long as 25-30 base pairs in length. They can be designed to anneal to the wild-type gene sequence in regions that flank a mutation in a gene of interest, such that extension from the primer amplifies a region that allows the detection of the presence or absence of a mutation of interest. Primers can also be designed such that their extension results in an amplified sequence only in the presence of either a wild-type or mutant gene, as desired. This can be accomplished by designing a primer with at least one nucleotide at the 3′end that is mismatched with the wild-type sequence, but matched to a mutant sequence. The invention is exemplified by primer pairs used to screen HNF-1α, glucokinase, amylin and human mitochondrial DNA for mutations. Of particular interest are nucleic acid primers that can be used to screen mutations in HNF-1α and glucokinase that have not yet been previously reported (see for example, SEQ ID Nos: 34-36). Simultaneous sequencing of several nucleic acid samples can also be carried out on a microchip (see U.S. Pat. No. 6,197,506)

For SSCP, primers are designed that amplify DNA products of about 250-300 bp in length across non-duplicated segments of the gene of interest. For each amplification product, one gel system and two running conditions are used. Each amplification product is applied to a 10% polyacrylamide gel containing 10% glycerol. Separate aliquots of each amplimer are subjected to electrophoresis at 8 W at room temperature for 16 hours and at 30 W at 4° C. for 5.4 hours. These conditions were previously shown to identify 98% of the known mutations in the CFTR gene (Ravnik-Glavac et al, (1994) Hum Mol Genet 3: 801).

As with identification of associative mutations of interest, identification of associative SNPs that correlate with the increased risk of a Chinese individual to develop type 2 diabetes can be accomplished by nucleic acid sequencing of desired regions of genomic or complementary DNA. Screening for SNPs is pursued most efficiently using microarray technologies where attached nucleic acid sequences attached to a solid support such as a microchip are exposed to hybridization conditions that allow the discrimination between two nucleic acid sequences that differ at one nucleotide (see for example, Wang, et al (1998) Science 280: 1077; and Hacia, et al (1998) Nature Genet. 18: 155). Alternatively, mass spectrophotometers can be used to identify small mass differences in PCR products that have single nucleotide polymorphisms (see Kirpekar, et al (1998) Nucleic Acids Res 26: 2554). A further means of analyzing genetic information is” dynamic allele specific hybridization” (DASH). This technique uses labeled oligonucleotides in a multiwell format that will fluoresce when the oligonucleotide exists in a double-stranded form, but not when it is in single-stranded form. Adding a single strand of the DNA to be tested allows the strands to hybridize. The temperature at which the strands denature will allow identification of the base at the SNP. The DASH technique has the advantages of being technically simple, and not requiring expensive equipment. Additional techniques that can be used in the screening for SNPs associated with the genetic predisposition of a Chinese person to develop type 2 diabetes include exonuclease resistance, microsequencing, solution-phase or solid phase extension of ddNTPs, and oligonucleotide ligation assay (as described in U.S. Pat. No. 5,952,174, herein incorporated by reference).

After the presence of an associative mutation or SNP is detected by any of the above techniques, the specific nucleic acid alteration comprising the mutation is identified by direct DNA sequence analysis or restriction analysis or a combination of both. In this manner, previously unidentified mutations in genes that encode proteins involved in insulin secretion, or in genes associated with obesity or diabetic kidney disease may be defined. For instance, new mutations could be identified with other genes known to closely correlate with familial type 2 diabetes in Chinese subjects (e.g., other MODY genes). Examples of additional MODY genes include hepatocyte nuclear factor 4α (HNF-4α), hepatocyte nuclear factor 1β (HNF-1β), and insulin promoter factor 1 (IPF-1). Additional candidate genes of particular interest for screening because mutations or polymorphisms of the wild type genes are positively associated with type 2 diabetes and nephropathy in Chinese individuals include those that encode angiotensin converting enzyme (ACE)/angiotensinogen (AGT) (Tomino, et al (1999) Nephron 82: 139; Hsieh, et al (2000) Nephrol Dial Transplant 15: 1008; Thomas, et al (2001) Diabetes Care 24; 356), aldose reductase (Ko, et al (1995) Diabetes 44: 727; Moczulski, et al (1999) Diabetologia 42: 94) and plasminogen activator inhibitor-1 (PAI-1) (Wong, et al (2000) Kidney Int 57: 632).

Nucleic acid sequences that encode genes involved with glucose metabolism, insulin resistance, obesity and diabetic kidney can also be screened to identify mutations in, for example, proteins that influence insulin binding to its receptor, that are involved in the insulin signaling pathway, that influence glucose uptake and cell metabolism. Specific examples include associative mutations in the α or β chain of the insulin receptor, the insulin receptor substrate proteins (IRS-1 and IRS-2), glucose transporter proteins GLUT2 and GLUT4, and transcription factors HNF-3β and NeuroDI/Beta2 and to correlated any identified mutation an/or polymorphism with indidence of type 2 diabetes. Examples of candidate genes where mutations or polymorphisms have been shown to be associated with type 2 diabetes and obesity in other populations include genes that encode the transporter GLUT4 (Abel, et al (2001) Nature 409: 729), the beta-3-adrenergic receptor (Oeveren van-Dybicz, et al (2001) Diabetes Obes Metab 3: 47), the hormone resistin (Steppan, et al2001) Nature 409: 307), the peroxisome proliferator-activated receptor gamma2 PPARgamma) (Hasstedt, et al (2001) J Clin Endocrinol Metab 86: 536), uncoupling protein-1 (UCP-1) (Heilbronn, et al (2000) Diabetologia 43: 242), leptin (Ohshiro, et al (2000) J Mol Med 78: 516), G protein beta 3 subunit and insulin receptor substrate-1 (Rosskopf, et al (2000) 5: 484), and the dopamine D2 receptor (Jenkinson, et al (2000) Int J Obes Relat Metab Disord 24: 1233). Additionally, mutations or polymorphisms shown to be closely associated with type 2 diabetes and nephropathy in other populations include genes that encode the G protein beta 3 subunit (Beige, et al (2000) Nephrol Dial Transplant 15: 1384; Zychma, et al (2000) Am J Nephrol 20: 305), methylenetetrahydrofolate reductase (MTHFR) (Shpichinetsky, et al (2000) J Nutr 130: 2493, the glucose transporter GLUT1 (Grzeszczak, et al (2001) Kidney Int 59: 631), and paraoxonase (PON1) (Inoue, et al (2000) Metabolism 49: 1400).

Additionally, proteins translated from genes carrying at least one mutation associated with increased risk of a Chinese individual to develop type 2 diabetes are contemplated by the invention and find use in functional diagnostic assays and in the production of diagnostic antibodies that bind to the mutant but not the wild-type protein. The polypeptides may be the translational products of the entire mutant gene, as well as peptides of twelve or more amino acids derived therefrom that contain at least one mutation of interest. The polypeptide (s) may be isolated from human tissues obtained by biopsy or autopsy, or may be produced in a heterologous cell by recombinant DNA methods, well known to those in the art (as disclosed in Molecular Cloning, A Laboratory Manual (2nd Ed., Sambrook, Fritsch and Maniatis, Cold Spring Harbor, 1989), or Current Protocols in Molecular Biology (Eds. Aufubel, Brent, Kingston, More, Feidman, Smith and Stuhl,Greene Publ. Assoc., Wiley-Interscience, NY, N.Y., 1992), both references herein incorporated by reference). Peptides comprising HNF-1α-, glucokinase- or amylin specific sequences may be derived from isolated larger polypeptides described above, using proteolytic cleavages by e. g. proteases such as trypsin and chemical treatments such as cyanogen bromide that are well-known in the art. Alternatively, peptides up to 60 residues in length can be routinely synthesized in milligram quantities using commercially available peptide synthesizers.

Recombinant translational products are expressed from vectors comprising mutant nucleic acid sequences of wild-type nucleic acid sequences that encode proteins involved in insulin secretion. Exemplified mutant nucleic acid sequences of interest include those that encode HNF-1α, glucokinase or amylin with single amino acid residue changes, as depicted in SEQ ID Nos: 1-13, and particularly SEQ ID NO: 2, SEQ ID NO: 7 and SEQ ID NO: 10. A large number of vectors, including plasmid and fungal vectors, have been described for expression in a variety of eukaryotic and prokaryotic hosts, and may be used for gene therapy as well as for simple protein expression. Vectors used for expression will also include a promoter operably linked to the mutant polypeptide encoding portion, that is preferably the cDNA sequence of the mutated gene of interest or a part thereof that encodes a polypeptide of at least 12 amino acids. The encoded polypeptide may be expressed by using any suitable commercially available vectors, and any suitable host cells, using methods disclosed or cited herein or otherwise known to those skilled in the relevant art. The particular choice of vector/host is not critical to the operation of the invention.

Appropriate host cells include bacteria, archebacteria, fungi, especially yeast, and plant and animal cells, especially mammalian cells. Of particular interest are E.coli, B.Subtilis, Saccharomyces cerevisiae, SF9 cells, C129 cells, 293 cells, Neurospora, and CHO cells, COS cells, HeLa cells, and immortalized mammalian myeloid and lymphoid cell lines. Preferred replication systems include M13, ColEI, SV40, baculovirus, lambda, adenovirus, and the like. A large number of transcription initiation and termination regulatory regions have been isolated and shown to be effective in the transcription and translation of heterologous proteins in the various hosts. Examples of these regions, methods of isolation, manner of manipulation, etc. are known in the art. Under appropriate expression conditions, host cells can be used as a source of recombinantly produced mutant polypeptides of interest.

The translational products of mutant HNF-1α, glucokinase or amylin, and/or fragments or portions thereof may be used to produce specific antibodies. The antibodies may be polyclonal or monoclonal, may be produced in response to the fully translated mutant polypeptide or to synthetic peptides as described above. Such antibodies are conveniently made using the methods and compositions disclosed in Harlow and Lane, Antibodies, A Laboratory Manual, Cold Spring Harbor Laboratory, 1988, other references cited herein, as well as immunological and hybridoma technologies known to those in the art. Importantly, the antibodies raised against translation products from nucleic acids sequences carrying at least one mutatation associated with type 2 diabetes should distinguish between the mutant amino acid sequence and the wild-type amino acid sequence. In particular, antibodies should have very little or no cross-reactivity for the wild-type sequence. Preferably the anti-mutant protein antibodies should bind with higher affinity to the mutant polypeptide than to wild-type polypeptide, with binding to the mutant polypeptide at levels 500:1, more preferably 1,000:1, greater than binding the wild-type polypeptide.

Isolated polypeptides corresponding to the entire length of the mutant polypeptide or a peptide of at least 12 amino acids in length containing a mutation of interest may be used in accordance with conventional methods to immunize a mammal, (e. g., mouse or higher mammal, primate, or chimeric or transgenic animals which produce human immunoglobulins) in accordance with conventional procedures. See for example, U.S. Pat. Nos. 4,172,124; 4,350,683; 4,361,549; and 4,464,465. Hybridomas may be prepared by fusing available myeloma lines, e. g., NS/I, Ag8.6.5.3, etc., with peripheral blood lymphocytes, splenocytes or other lymphocytes of the immunized host and the resulting immortalized B-lymphocytes (e.g., hybridomas, heteromyelomas, EBV transformed cells, etc.) selected, cloned and screened for binding to a mutant polypeptide of a wild-type protein involved in insulin secretion or glucose metabolism. Monoclonal antibodies raised against a mutant polypeptide sequence of interest may be of any immunoglobulin class such as IgA, IgD, IgE, IgG and IgM, preferably IgG or IgM, and may be of any one of the subclasses of the classes. Whole antibodies, or fragments thereof which retain binding activity, may be employed, such as Fab, F(ab′)₂, or the like. Once the antibodies with binding specificity for the mutant polypeptide are available, these antibodies may be used for screening. Antibodies that distinguish between normal and mutant forms of HNF-1α, glucokinase, amylin or other mutant/wild-type pairs of proteins involved in insulin secretory function may be used in diagnostic tests employing ELISA, EMIT, CEDIA, SLIFA, and the like.

For an assessment of total risk of developing disease or in designing individualized treatments of diagnosed patients, identified mutations and polymorphisms that are indicative of a Chinese individual to develop type 2 diabetes are correlated with phenotypic parameters of screened patients and interpreted with consideration of a positive or negative family history of the disease. Genetic studies will be correlated with data from individuals indicating hormone levels (growth hormone, adrenaline, cortisol, noradrenaline, insulin), anthropometry (body-mass index; waist-to-hip ratio), hemodynamics (blood pressure), cardiovascular risk factors (HDL, LDL, cholesterol, triglycerides) and autoimmunity (anti-glutamic acid decarboxylase antibodies). For instance, a patient with a single mutation in the glucokinase gene may never develop symptoms, whereas the likelihood of a patient with a mutation in both the glucokinase gene and the HNF-1α gene or a mutation in the glucokinase gene and the phenotypic attribute of obesity to develop overt type 2 diabetes is relatively higher. A positive family history of the disease would increase the predicted predisposition even more. Obtaining a genotypic assessment while a patient shows no signs of developing disease, or while showing preliminary signs of disease such as impaired glucose tolerance (IGT), can enable a physician to initiate therapy or suggest lifestyle changes that prevent the onset or progression of overt symptoms. For example, a patient identified as having a mutation in the HNF-1α gene and IGT, can be treated with diet and/or oral drugs and/or insulin early enough that hyperglycemic toxicity of pancreatic β-cells and further insulin secretory dysfunction due to their death is prevented or ameliorated. In this way, severe complications associated with progressive type 2 diabetes, such as nephropathy, retinopathy and sensorineural loss, can be more commonly averted.

In addition to allowing a clinician to better tailor traditional therapies for treating type 2 diabetes, such as diet, oral drugs and insulin, identification of associative mutations can enable a clinician to design tailored therapies, such as introducing a wild-type gene into a patient to replace a mutant gene that encodes a malfunctioning protein. For gene therapy methods, transfection in vivo is obtained by introducing a therapeutic transcription or expression vector into the mammalian host, either as naked DNA, complexed to lipid carriers, particularly cationic lipid carriers, or inserted into a viral vector, for example a recombinant adenovirus. The introduction into the mammalian host can be by any of several routes, including intravenous or intraperitoneal injection, intratracheally, intrathecally, parenterally, intraarticularly, intranasally, intramuscularly, topically, transdermally, application to any mucous membrane surface, corneal installation, etc. Of particular interest is the introduction of a therapeutic expression vector into a circulating bodily fluid or into a body orifice or cavity, such as the heart. Thus, intravenous administration and intrathecal administration are of particular interest since the vector may be widely disseminated following such routes of administration, and aerosol administration finds use with introduction into a body orifice or cavity. Particular cells and tissues can be targeted, depending upon the route of administration and the site of administration. For example, a tissue which is closest to the site of injection in the direction of blood flow can be transfected in the absence of any specific targeting. An arterial catheter can be used to introduce the expression vector into an organ such as the heart or kidney. The eye can be accessed directly either by the use of ocular drops or by injecting into the eye. For accessing nerves, this can be by injection into the nerve or injection into the region of the cell body. If lipid carriers are used, they can be modified to direct the complexes to particular types of cells using site-directing molecules. Thus, antibodies or ligands for particular receptors or other cell surface proteins may be employed, with a target cell associated with a particular surface protein. An amino terminal mitochondrial targeting sequence joined to a nucleic acid can be used to target the nucleic acid to the mitochondria. See Taylor et al, Nature Genetics 15: 212-215,1997.

Any physiologically acceptable medium may be employed for administering the DNA, recombinant viral vectors or lipid carriers, such as deionized water, saline, phosphate-buffered saline, 5% dextrose in water, and the like as described above for the pharmaceutical composition, depending upon the route of administration. Other components may be included in the formulation such as buffers, stabilizers, biocides, etc. These components have found extensive exemplification in the literature and need not be described in particular here. Any diluent or components of diluents that would cause aggregation of the complexes should be avoided, including high salt, chelating agents, and the like.

The amount of therapeutic vector used will be an amount sufficient to provide for a therapeutic level of expression in a target tissue susceptible to diabetic complications or for adequate dissemination to a variety of tissues after entry into the bloodstream and to provide for a therapeutic level of expression in susceptible target tissues. A therapeutic level of expression is a sufficient amount of expression to prevent, treat, or palliate one or more diabetic complication or the symptoms of diabetic complications. In addition, the dose of the nucleic acid vector used must be sufficient to produce a desired level of transgene expression in the affected tissue or tissues in vivo. Other DNA sequences, such as adenovirus VA genes can be included in the administration medium and be co-transfected with the gene of interest. The presence of genes coding for the adenovirus VA gene product may significantly enhance the translation of mRNA transcribed from the expression cassette if this is desired.

A number of factors can affect the amount of expression in transfected tissue and thus can be used to modify the level of expression to fit a particular purpose. Where a high level of expression is desired, all factors can be optimized, where less expression is desired, one or more parameters can be altered so that the desired level of expression is attained. For example, if high expression would exceed the therapeutic window, then less than optimum conditions can be used.

The level and tissues of expression of the recombinant gene may be determined at the mRNA level as described above, and/or at the level of polypeptide or protein. Gene product may be quantitated by measuring its biological activity in tissues. For example, protein activity can be measured by immunoassay as described above, by biological assay such as inhibition of ROS, or by identifying the gene product in transfected cells by immunostaining techniques such as probing with an antibody which specifically recognizes the gene product or a reporter gene product present in the expression cassette.

Typically, the therapeutic cassette is not integrated into the patient's genome. If necessary, the treatment can be repeated on an ad hoc basis depending upon the results achieved. If the treatment is repeated, the patient can be monitored to ensure that there is no adverse immune or other response to the treatment.

The following examples are offered by way of illustration of the present invention, not limitation.

### EXPERIMENTAL

### EXAMPLE 1

### Identification of Mutations in Glucokinase and Hepatocyte Nuclear Factor 1α Genes in Chinese Patients with Early-Onset Type 2 Diabetes Mellitus/MODY

This example illustrates mutations identified in the glucokinase, HNF-1α and HNF-4α genes in a cohort of Chinese patients. Mutations in the glucokinase and HNF-1α genes are relatively common in early-onset diabetes and they account for about 3% and 5%, respectively, of the present Chinese early-onset diabetic patients.

Experimental Design and Methods

Subjects

The study group consisted of 92 unrelated patients (age 34±5 years (mean±SD) range 18-40 years; 30 males and 62 females) who were diagnosed with Type 2 diabetes before 40 years of age and who had a positive family history (at least one first degree relative with Type 2 diabetes). The mean age at diagnosis was 30±5 years (range 16-40 years). Thirteen (14%) of these patients met the minimal criteria of MODY (age at diagnosis before 25 years old and presence of diabetes in two consecutive generations). These patients were selected from a database containing 1800 cases recruited in the Diabetes and Endocrine Centre of the Prince of Wales Hospital. Family members of probands with MODY gene mutations, if available, were recruited and underwent a 75-gram oral glucose tolerance test (OGTT). One hundred healthy Chinese (age 33±10 years, 40 males and 60 females) without a history of diabetes were recruited as controls amongst hospital staff and students. Informed consent was obtained from each subject for a blood sample to be taken for DNA isolation and measurement of clinical parameters. This study was approved by the Clinical Research Ethics Committee of The Chinese University of Hong Kong.

Screening of Glucoknase, HNF-1α and HNF-4α Genes for Mutations

The minimal promoter region and exons of the glucokinase (β cell form), HNF-1α and HNF-4α (HNF-4α 2 form) genes were screened for mutations by direct sequencing of polymerase chain reaction (PCR) products as described (Froguel, et al (1993) N Engl J Med 328: 697; Yamagata, et al (1996) Nature 384: 455; Yamagata, et al (1996) Nature 384: 458). The occurrence of putative mutations in other family members and controls was determined by PCR-restriction fragment length polymorphism (RFLP). An artificial restriction site was introduced into either the wild-type or mutant sequence if the nucleotide substitution did not lead to gain or loss of a restriction site. Briefly, direct sequencing identified 5 mutations in the HNF-1α gene (G20R, R203H; S432C, I618M and IVS2nt-1G→A) and 3 mutations in the glucokinase gene (I110T, A119D and G385V) that are unique to Chinese subjects. They were screened as follows.

HNF-1α G20R was screened by using the forward primer 5′-GGCAGGC-AAACGCAACCCACG-3′ (SEQ ID NO:14) and modified reverse primer 5′-CAGTGCCTCTTTGCTCAGGC-3′ (SEQ ID NO:15) for PCR amplification followed by digestion with StuI. The wild-type allele showed 19 and 140 bp products whereas the mutant allele showed a 159 bp product.

HNF-1α R203H was screened by using the forward primer 5′-TGCCTGCAGAGTTCA-CCCATG-3′ (SEQ ID NO:16) and modified reverse primer5′-ATCTGCTGGGATGCTGGG-CCCCACTTGCAA-3′ (SEQ ID NO:17) for PCR amplification followed by digestion with BsrDI. The wild-type allele showed a 121 bp product whereas the mutant allele showed 26 and 95 bp products.

HNF-1α S432C was screened by using the forward primer 5′-TGGAGCAGTCCCTAG-GGAGGC-3′ (SEQ ID NO:18) and reverse primer 5′-GTTGCCCCATGAGCCTCCCAC-3′ (SEQ ID NO:19) for PCR amplification followed by digestion with Cac81. The wild-type allele showed 104 and 218 bp products whereas the mutant allele showed 37,67 and 218 bp products.

HNF-1α 1618M was screened by using the forward primer 5′-GTACCCCTAGGGACAGG-CAGG-3′ (SEQ ID NO:20) and reverse primer 5′-ACCCCCCAAGCAGGCAGTACA-3′ (SEQ ID NO:21) for PCR amplification followed by digestion with TaqI. The wild-type allele showed 88 and 160 bp products whereas the mutant allele showed a 248 bp product.

HNF-1α IVS2nt-1G→A was screened by using the forward primer 5′-GGGC-AAGGTCAGGGGAATGGA-3′ (SEQ ID NO:22) and reverse primer 5′-CAGCCCAGACCAAACCAGCAC-3′ (SEQ ID NO:23) for PCR amplification followed by digestion with PstI. The wild-type allele showed 73 and 231 bp products whereas the mutant allele showed a 304 bp product.

Glucokinase I110T was screened by using the forward primer 5′-GTCCCTGAGGCTG-ACACACTT-3′ (SEQ ID NO:24) and reverse primer 5′-AGCTGGGCCCTGAGATCCTGCA-3′ (SEQ ID NO: 25) for PCR amplification followed by digestion with FokI. The wild-type allele showed 108 and 142 bp products whereas the mutant allele showed a 250 bp product.

Glucokinase A 119D was screened by using the forward primer 5′-ACCTGGGTGGCA-CTAACTTCA-3′ (SEQ ID NO:26) and modified reverse primer 5′-CGGCCCCTGCGCTG-CTCACCATCTGA-3′ (SEQ ID NO:27) for PCR amplification followed by digestion with BclI. The wild-type allele showed a 150 bp product whereas the mutant allele showed 28 and 122 bp products.

Glucokinase G385V was screened by using the forward primer 5′-GGACTGTCG-GAGCGACACTCA-3′ (SEQ ID NO:28) and modified reverse primer 5′-GCGGTTGATGAC-GCCTGCCAG-3′ (SEQ ID NO:29) for PCR amplification followed by digestion with FauI. The wild-type allele showed 5,22,44 and 137 bp products whereas the mutant allele showed 5,44 and 159 bp products.

Mutations in the amylin gene (S20G) and mitochondrial DNA (A3243G) were screened as follows.

Amylin S20G was screened by using the forward primer 5′-TCACAT-TTGTTCCATGTTAC-3′ (SEQ ID NO:30) and reverse primer 5′-CAATAACTATAGAG-TTACATTG-3′ (SEQ ID NO:31) for PCR amplification followed by digestion with MspI. The wild-type allele showed a 239 bp product whereas the mutant allele showed 99 and 140 bp products.

Mitochondrial DNA A3243G was screened by using the forward primer 5′-AAGGTTCGTT-TGTTCAACGA-3′ (SEQ ID NO:32) and reverse primer 5′-AGCGAAGGGTTGTAGTAGCC-3′ (SEQ ID NO:33) for PCR amplification and labeling of PCR product with α³²PdATP at the last cycle. The PCR products were then digested with ApaI and analysed on 8% denaturing polyacrylamide gels. The wild-type allele showed a 427 bp product whereas the mutant allele showed 213 and 214 bp products.

Clinical Studies

All patients underwent a structured assessment including documentation of family history, age at diagnosis and body mass index (BMI) (Piwernetz, et al (1993) Diabetic Med 10: 371; Chan, et al (1997) Hong Kong Auth Qual Bull 2: 3). Family history was documented in two generations only since the diabetic status of grandparents was usually unknown. A fasting blood sample was taken for the measurement of glucose, C-peptide and glycosylated haemoglobin (HbA_{1c}). Obesity was defined as a BMI≧27 kg/m²in men and ≧25 kg/m²in women (National Diabetes Data Group (1979) Diabetes 28: 1039).

Assays

Plasma glucose concentrations were measured by a glucose oxidase method (Diagnostic Chemicals, Charlottetown, Prince Edward Island, Canada). C-peptide was measured by radioimmunoassay (Novo-Nordisk, Copenhagen, Denmark). HbA_{1c} was measured by gel electrophoresis (Ciba Coming Diagnostics Corp, Palo Alto, Calif.).

Data Analysis

Data are expressed as mean ±SD if normally distributed. Otherwise, data are expressed as median and range.

Results

Mututions and Polymorphisms in the Glucokinase, HNF-1_{α} and HNF-4_{α} Genes

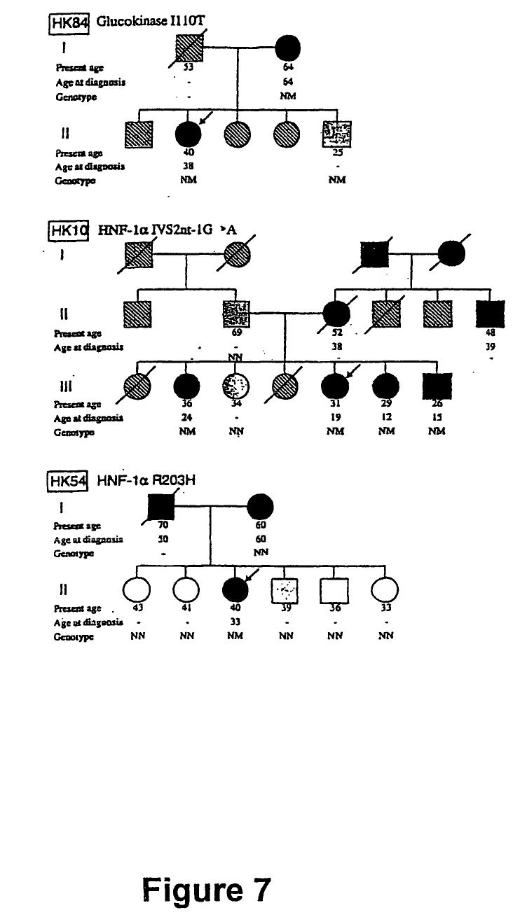

Screening of the promoter region and exons 1a, 2-10 of-the glucokinase gene (Stoffel, et al (1992) Proc Nratl Acad Sci 89: 7698; Tanizawa, et al (1992) 6:1070) revealed three novel missense mutations: I110T, A119D and G385V. In addition to these mutations, three uncommon variants (two of which had not been previously described) and two polymorphisms were found in the 5′-untranslared region of the mRNA and intron regions (Table 1). The brother and mother of subject HK84 (Table 1) also inherited the I110T mutation (FIG. 7). The mother was diagnosed with diabetes at the age of 64 years upon screening. The brother aged 25 years, when tested with a 75 g OGTT had a plasma glucose at 0 and 120 min of 6.3 mmol/1 and 6.9 mmol/1, respectively. These results were inconclusive, suggesting impaired fasting glucose (IFG) by the 1997 ADA criteria but not reaching that of impaired glucose tolerance (IGT) by the 1998 WHO criteria.

Screening of the HNF-1α gene revealed four missense mutations (G20R, R203H, S432C and 1618M) and one splice acceptor site mutation (IVS2nt-1G→A) (Table 2). All of these represent mutations in the HNF-1α gene unique to Chinese patients. Subject HK10 (Table 2) had three siblings (ages 26-36 years) with diabetes. The affected siblings all had inherited the IVS2nt-IG→A mutation while another sibling and the father with IGT had not. Moreover, the maternal grandparents, uncle and mother of HK10 were diabetic but they were not available for screening (FIG. 7) (Chan, et al (1990) Diabetic Med 7: 211). Subject HK54 (Table 2) had four siblings (age 33-43 years) with normal glucose tolerance and one sibling (age 39 years) with IGT. The father and mother were diagnosed as having diabetes at the ages of 50 and 60 years, respectively. Neither the mother and nor any of the siblings had inherited the R203H mutation (FIG. 7). In addition to the putative diabetes-associated mutations in HNF-1α two substitutions resulting in common amino acid polymorphisms, four silent mutations and nine variants/polymorphisms in introns were identified (Table 2). Family members of the other five probands (Tables 1 and 2) with glucokinase or HNF-1α missense mutations were not available for screening. None of the mutations in the glucokinase and HNF-1α genes were found in 100 healthy controls.

Analysis of the promoter region and exons 1a, 2-10 of the HNF-4α gene (Furuta, et al (1997) Diabetes 46: 1652) revealed no obvious diabetes-associated mutations. Three patients were heterozygous for a previously described amino acid polymorphism, T/1130 (Yamagata et al, (1997) Nature 384: 458). Two patients were heterozygous for a silent mutation in the codon for L211, and one patient was heterozygous for a silent mutation in the codon for P441 (Table 3). There were two polymorphisms in the intron-upstream of exon 2 (intron 1B) and a G→A substitution in the promoter was found in the heterozygous state in one patient. The G→A substitution in the promoter at nucleotide-462 was not located in a known cis-acting regulatory region of the gene (Furuta, et al (1997) supra) and its effect on the regulation of expression of HNF-4α remains to be determined.

Clinical Features of Patients with MODY or Unknown Etiology

The clinical features of the patients with mutations in the glucokinase and HNF-1α genes or with unknown etiology are shown in Table 4. Of the 92 patients, 54 (59%) were non-obese at the time of study. The mean age at diagnosis of the patients with glucokinase mutation-associated diabetes (‘glucokinase diabetes’) was 28 years. All three subjects had mild hyperglycemia and satisfactory glycemic control (fasting glucose ≦7.4 mmol/1; HbA_{1c}, 6.7%; non-diabetic range: 5.1-6.4%). These patients had varving degrees of basal pancreatic β cell secretory function as indicated by their fasting C-peptide levels (0.28-1.60 nmol/1) (Chan, et al (1990) supra). All were treated with diet or oral drugs. No diabetic complications were observed in the three patients with glucokinase mutation (Froguel, et al (1993) supra ; Page, et al (1995) 12: 209; Velho, et al (1997) 40: 217).

The mean age at diagnosis of the patients with HNF-1α mutation-associated diabetes was 31 years. Among the four patients (HK30, 54,90 and 92) with missense mutations, all had mild hyperglycemia and satisfactory glycemic control (fasting glucose ≦7.4 mmol/1; HbA_{1c}≦7.1%) but exhibited varying degrees of basal pancreatic β cell secretory function (fasting C-peptide, 0.10-0.49 nmol/1). They did not have diabetic complications and were treated with diet or oral drugs. The subject (HK10) with the splice-site IVS2nt-1G→A mutation was not overweight when diagnosed at the age of 19 years (Fajans (1990) Diabetes Care 13: 49-64) and presented with proliferative retinopathy and clinical proteinuria. She was treated with insulin continuously for three months after the diagnosis. She eventually developed neuropathy and renal failure.

TABLE 1
Mutations and polymorphisms in the glucokinase gene in
Chinese subjects with early-onset Type 2 diabetes mellitus

| | | | | | |
|---|---|---|---|---|---|
| Subject | Location | Codon/nt | Nucleotide change | Designation | Frequency |

Mutations

| | | | | |
|---|---|---|---|---|
| HK84 | Exon 3 | 110 | ATC (Ile)→ACC (Thr) | I110T |
| HK38 | Exon 3 | 119 | GCT(Ala)→GAT(Asp) | A1I9D |
| HK15 | Exon 9 | 385 | GGG(Gly)→GTG(Val) | G385V |

Polymorphisms

| | | | | |
|---|---|---|---|---|
| 5′-UT* | −213 | A→G | 5′-UT β − 213 A/G | A 0.96, G 0.04 |
| 5′-UT | −84 | C→G | 5′-UT β − 84 C/G | C 0.94, G 0.06 |
| Intron 1c | nt − 13 | C→G | IVS1nt − 13 C/G | C 0.99, C 0.01 |
| Intron 9 | nt + 8 | C→T | IVS9nt + 8C/T | C 0.50, T 0.50 |
| Intron 9* | nt + 49 | G→A | IVS9nt + 49G/A | G 0.99, A 0.01 |

nt indicates the nucleotide location relative to the first nucleotide of codon 1 (ATG) for polymorphisms in the 5′-untranslated region (5′-UT) of the β cell specific exon 1 α/1 β, and splice donor (+) or acceptor site (−).
Intron 1c is the intron between exon 1c, which encodes the amino terminal 14 amino acids of the minor liver isoform of glucokinase, and exon 2 (Velho, et al (1996) 19: 915).
The asterisks indicate polymorphisms that were reported by Ng, et al (Diabetic Med (1999) 16: 956) and that have not been reported in studies of other populations (Veiga-deCunha, et al (1996) J Biol Chem 271: 6292; Zhang, et al (1995) 38: 1055).

TABLE 2
Mutations and polymorphisms in the HNF-1 α gene in Chinese subjects
with early-onset Type 2 diabetes mellitus

| | | | | | |
|---|---|---|---|---|---|
| Subject | Location | Codon/n | Nucleotide change | Designation | Frequency |

Mutations

| | | | | |
|---|---|---|---|---|
| HK90 | Exon 1 | 20 | GGG (Gly)→AGG (Arg) | G20R |
| HK10 | Intron 2/ | nt − 1 | AG→AA at splice | IVS2nt − 1G→A |
| | Exon 3 | | acceptor site | |
| HK54 | Exon 3 | 203 | CGT (Arg)→CAT (His) | R203H |
| HK30 | Exon 6 | 432 | TCC (Ser)→TGC (Cys) | S432C |
| HK92 | Exon 10 | 618 | ATC (Ile)→ATG (Met) | I618M |

Silent mutations/polymorphisms

| | | | | |
|---|---|---|---|---|
| Exon 1 | 17 | CTC (Leu)→CTG (Leu) | L17C/G | C 0.63, C 0.37 |
| Exon 1 | 27 | ATC (Ile)→CTC (Leu) | I/L27 | A 0.57, C 0.43 |
| Intron 1 | nt − 42 | G→A | IVS1nt − 42G/A | C 0.58, A 0.42 |
| Intron 2* | nt + 53 | C→G | IVS2nt + 53C/G | C 0.99, C 0.01 |
| Intron 2 | nt − 51 | T→A | IVS2nt − 51T/A | T 0.77, A 0.23 |
| Intron 2 | nt − 23 | C→T | IVS2nt − 23C/T | C 0.48, T 0.52 |
| Intron 5 | nt + 9 | C→G | IVS5nt + 9C/G | C 0.98, G 0.02 |
| Intron 5 | nt − 42 | G→T | IVS5nt − 42G/T | G 0.87, T 0.13 |
| Intron 6* | nt + 26 | C→T | IVS6nt + 26C/T | C 0.99, T 0.01 |
| Exon 7 | 459 | CTG (Leu)→TTG(Leu) | L459C/T | C 0.48, T 0.52 |
| Exon 7 | 459 | CTG (Leu)→CTA (Leu) | L459G/A | C 0.99, A 0.01 |
| Exon 7 | 487 | AGC (Ser)→AAC (Asn) | S/N487 | G 0.48, A 0.52 |
| Intron 7 | nt + 7 | G→A | IVS7nt + 7G/A | G 0.48, A 0.52 |
| Exon 8* | 531 | AGC (Ser)→AGT (Ser) | S531 C/T | C 0.99, T 0.01 |
| Intron 9 | nt − 24 | T→C | IVS9nt − 24T/C | T 0.48, C 0.52 |

nt indicates the nucleotide location relative to the splice donor (+) or acceptor site (−).
The asterisks indicate polymorphisms reported by Ng, et al (Diabetic Med (1999) 16: 956) and that have not been reported in studies of other populations.

TABLE 3
Mutations and polymorphisms in the HNF-4 α gene in Chinese subjects with
early-onset Type 2 diabetes mellitus

| Location | Codon/nt | Nucleotide change | Designation | Frequency |
|---|---|---|---|---|
| Silent mutations/ | | | | |
| polymorphisms | | | | |
| Promoter* | nt-462 | G→A | Ptr-462G/A | G 099, A 0.01 |
| Intron lB | nt-38 | C→T | IVS1nt-38C/T | C 0.80, T 0.20 |
| Intron lB | nt-5 | C→T | IVS1nt-5C/T | C 0.79, T 0.21 |
| Exon 4 | 130 | ACT (Thr)→ATT (Ile) | T/I130 | C 0.98, T 0.02 |
| Exon 6* | 211 | CTC (Leu)→CTT (Leu) | L211C/T | C 0.99, T 0.01 |
| Exon. 10* | 441 | CCG (Pro)→CCA (Pro) | P441G/A | G 0.99, A 0.01 |
| | | | | G 0.99, A 0.01 |

nt indicates the nucleotide location relative to the first nucleoride of codon 1 (ATG) for polymorphisms in the promoter region and splice donor (+) or acceptor site (−).
The sequence context of the nt-464 polymorphism is GATA(G/A)TATC.
The asterisks indicate polymorphisms that have not been reported in studies of other populations.

TABLE 4
Clinical features of Chinese patients with early-onset Type 2 diabetes
of unknown etiology as compared with those with diabetes as a
result of mutations in glucokinase and HNF-1 α genes

| | Unknown | Glucokinase | | | | | | | |
|---|---|---|---|---|---|---|---|---|---|
| | Etiology | diabetes (n = 3) | | | HNF-1 α diabetes (n = 5) | | | | |
| | (n = 84) | HK1 | HK38 | HK84 | HK10 | HK30 | HK54 | HK90 | HK92 |
| Age at diagnosis (year) | 30 ± 5 | 18 | 29 | 38 | 19 | 30 | 33 | 36 | 38 |
| Interval since diagnosis (year) | 3 (0-16) | 15 | 2 | 1 | 9 | 3 | 6 | 0 | 0 |
| Sex(M/F)(%) | 32/68 | F | F | F | F | M | F | M | M |
| Family history(fa/mot/sib)(%) | 45/63/25 | fa | mot, sib | mot | gparent, | fa | fa, mot | mot, sib | mot |
| | | | | | uncle, | | | | |
| | | | | | mot, sib | | | | |
| BMI (kg/m²) | 26 ± 5 | nd* | 15 | 28 | 26 | 23 | 20 | 20 | 29 |
| HbA_{1c}(%) | 7.5 ± 1.9 | 6.7 | 6.0 | 6.6 | 8.7 | 7.1 | 6.0 | 6.9 | 6.1 |
| Fasting glucose (mmol/l) | 8.5 ± 3.3 | 7.2 | 7.4 | 6.6 | 13.9 | 7.4 | 4.9 | 4.9 | 6.6 |
| Fasting C-peptide (nmol/l) | 0.43 (0.03-4.96) | nd | 1.60 | 0.28 | 0.47 | 0.49 | 0.11 | 0.16 | 0.10 |
| Treatment (D/O/I)(%) | 49/43/8 | O | O | D | I | O | O | D | O |

gparent, grandparent affected;
uncle, uncle affected;
fa, father affected;
mot, mother affected;
sib, siblings affected;
D, diet;
O, oral drugs;
I, insulin;
nd, not done.
Data are expressed as mean ± SD median (range) or n.
*BMI was not measured because this patient had spinomuscular atrophy.

The mean age at diagnosis of the 84 patients with unknown etiology was 30 years, similar to those with glucokinase or HNF-α gene mutations. Large variations in the degree of hyperglycemia (fasting glucose 8.5±3.3 mmol/1) and basal pancreatic β cell secretory function (fasting C-peptide 0.03-4.96 nmol/1) were observed. Most of these pateints were treated with oral drugs or diet (92%).

### EXAMPLE 2

### Mitochondrial DNA A3243G Mutation in Patients with Early-or Late-Onset Type 2 Diabetes Mellitus in Hong Kong Chinese

This example illustrates the prevalence of the mitochondrial DNA A3243G mutation in the Hong Kong Chinese population as represented by a large cohort of type 2 diabetic patients with differing ages of diagnosis and clinical phenotypes.

Experimental Design and Methods

Subjects

The study group consisted of 906 unrelated type 2 diabetic patients diagnosed according to the 1985 WHO criteria (World Health Organization, 1985). This cohort included four groups of patients selected according to the age of diagnosis and the presence or absence of family history of diabetes. Groups 1 and 2 consisted of 219 and 128 patients, respectively, with an early age of diagnosis (≦40 years) and with (Group 1) or without (Group 2) a family history of diabetes. Groups 3 and 4 consisted of 211 and 348 patients, respectively, with an older age of diagnosis (>40 years) and with (Group 3) or without (Group 4) a family history of diabetes. Patients in each of these groups were randomly selected from a cohort recruited in the Diabetes and Endocrine Centre of the Prince of Wales Hospital, which has a catchment of 1.2 million population in Hong Kong. All the patients underwent a structured assessment based on the Europe DiabCare Protocol (Piwernetz, et al (1993) Diabetic Med 10: 371; Chan, et al (1997) Hosp Auth Qual Bull 2: 3). Family members of mt3243 mutation carriers, if available, were recruited and underwent a 75 grain oral glucose tolerance test (OTT) Two hundred and thirteen healthy Chinese without a history of diabetes were recruited as control subjects amongst hospital staff and students. The present study group included 75 early onset type 2 diabetic (two of whom had an mt3243 mutation) and 95 control subjects who were included in a previous report (see Smith, et al (1997) Diabetic Med 14: 1026). Informed consent was obtained from each subject for a blood sample to be taken for DNA isolation and measurement of clinical partners. This study was approved by the Clinical Research Ethics Committee of The Chinese University of Hong Kong.

Mt3243 Mutation Analysis

Leukocyte DNA was extracted by standard methods involving proteinase K and phenol/chloroform (Sambrook, et al (1989) Molecular Cloning: A Laboratory Manual,Cold Spring Harbor Laboratory Press, New York, incorporated herein by reference). The mt3243 genotype was determined by polymerase chain reaction (PCR) amplification and ApaI digestion as described (Smith, et al, (1997) supra). In brief, the DNA region spanning nucleotide 3029 and 3456 was amplified by PCR and labelled with α³²PDATP at the last cycle. This method prevents the underestimation of the proportion of mutant mtDNA as a consequence of heteroduplex formation during the PCR (Schoffner, et al (1990) Cell 61: 931). The PCR products were then digested with ApaI (Gibco BRL, Gaithersburg, Md., USA) for 2 h at 30 C. Digested PCR products were electrophoresed on 8% denaturing polyacrylamide gels and visualized by autoradiography. The presence of mt3243 led to the cleavage of the 427 bp product into 213 and 214 bp fragments. Standards containing 0-100% mutant mt3243 (made by mixing a cloned DNA carrying no mt3243 mutation and another cloned DNA carrying >99% mutant mt3243 in different proportions, kindly given by Dr J. van den Ouweland, Leiden University) were also included in the assay. The 100% mutant DNA was used as a positive control to evaluate completeness of PCR product digestion. The intensity of bands was quantified by a Bio-Rad Model GS-670 imaging densitometer and a Molecular Analyst software (version 1.3) (Bio-Rad, Hercules, Calif., USA). The proportion of mt3243 in a sample was calculated by dividing the intensity at mutant bands (213 and 214 bp) by the total intensity of both wild-type and mutant bands.

Clinical Studies

All patients underwent a structured assessment including documentation of family history, age of diagnosis, body mass index (BMI) and waist-to-hip ratio. Audiometry was performed by a technician at the otolaryngology department to assess the sensorineural status in subjects carrying the mt3243 mutation. A fasting blood sample was taken for the measurement of glucose, C-peptide, insulin and glycosylated haemoglobin (HbA_{1c}). Insulin resistance (IR) was estimated using the homeostasis model assessment (HOMA) where IR=fasting insulin×fasting glucose/22.5 (Matthews et al, (1985) Diabetologia 28: 412). Obesity was defined as a BMI≧27 kg/m² in men and ≧25 kg/m² in women (National Diabetes Data Group, 1979). The basal pancreatic β-cell reserve was also assessed by plasma fasting C-peptide level. Patients with a C-peptide level≦0. 2 nmol/l were considered to be insulin deficient (Service et al,. (1997) Diabetes Care 20: 198).

Biochemical Assays

Plasma glucose was measured by a glucose oxidase method (Diagnostic Chemicals, Charlottetown, PEI, Canada). HbA_{1c} was measured by gel electrophoresis (Ciba Coming Diagnostics Corp, Palo Alto, Calif. USA) C-peptide was measured by radioimmunoassay (Novo-Nordisk, Copenhagen, Denmark) with an intra-assay coefficient of variance (CV) of 3.4% and interassay CV of 9.6%. Insulin was measured by radioimmunoassay (Pharmacia, Uppsala, Sweden) with in intra-assay CV of 6% and interassay CV of 13.8%.

Statistical Analysis

Data are expressed as mean ±SD or median (range) as appropriate. The X² test was used for analysing categorical data. Spearman correlation was used for measurement of association between variables. All statistics was performed with the Statistical Package for Social Sciences (SPSS) for Windows, version 6.1. A P-value<0.05 was considered as significant.

Results

The clinical details of the 906 type 2 diabetic patients are shown in Table 5. A significantly higher prevalence of maternal over paternal history of diabetes was found in both early- (Group 1) and late-onset (Group 3) diabetic patients with a positive family history (Table 5).

Amongst the 906 type 2 diabetic patients, in addition to the two patients reported previously (Smith, et al (1997) supra), three more patients carrying the mt3243 mutation were identified. In Group 1, this mutation was found in 1.8% of (four of 219) early onset patients with a positive family history. This prevalence increased to 3% (four of 133) if only those with a positive maternal family history were considered. In addition, one of the 348 late-onset patients without a family history (Group 4) was found to have this mutation (0.3%). None of the 128 early onset patients who had no family history (Group 2) or 211 late-onset patients with a positive family history (Group 3) or 213 control subjects had the mutation.

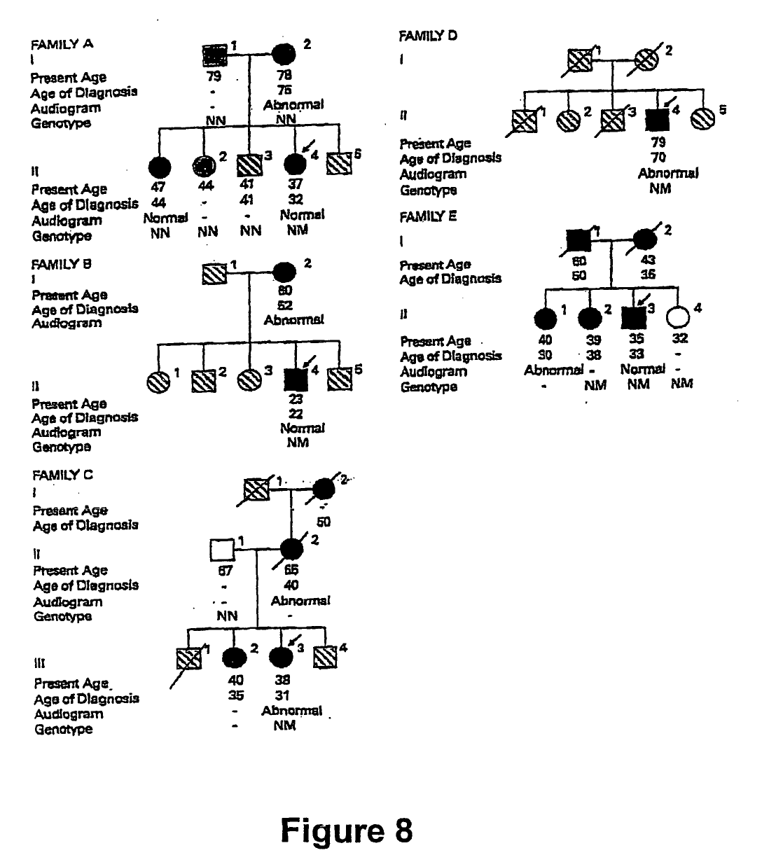

Amongst the five probands with the mt3243 mutation, three families were recruited (FIG. 8). In family A, family members with diabetes or IGT were identified but none of them carried the mutation. In Family E, two more subjects were found to have the mutation of whom one had diabetes. The clinical and biochemical characteristics of subjects carrying the mt3243 mutation are summarized in Table 6.

The percentage of mt3243 varied from 1% to 14% (Table 6). There was no correlation of heteroplasmy level of mutation with levels of HbA_{1c} fasting plasma glucose, C-peptide, insulin, insulin resistance or the presence of sensorineural impairment (P>0.05) (Table 6).

Families A and B

These two families have been reported in a previous study (Smith, et al, (1997) supra). The 37-year-old proband (II 4) in family A had been treated with oral drugs since diagnosed at the age of 32 years. The mother (I-2) and two siblings (II-1 and II-3) were diabetic while the father (I-1) and one sister (II-2) had IGT. However, none of the family members had the mt3243 mutation although the mother had a history of hearing loss.

The proband of family B was diagnosed as having diabetes at the age of 22 years and had been treated with oral drugs. The mother was diabetic and deaf but was not available for screening.

Family C

The 38-year-old proband (III-3) was diagnosed with diabetes at the age of 31 years. She was treated with diet and oral drugs for 6 years before being changed to insulin therapy. Audiometry revealed bilateral high tone sensorineural impairment. The father (II-1) had normal glucose tolerance and did not have the mt3243 mutation. The older sister (III-2) and the mother (II-2) both developed diabetes at about 40 years of age and the grandmother had diabetes at the age of 50 years. The mother became deaf at the age of 59 years. None of these affected members were available for screening.

TABLE 5
The clinical and biochemical features of 902 Chinese patients with type 2 diabetes

| | Group 1 | Group 2 | Group 3 | Group 4 |
|---|---|---|---|---|
| | (n = 219) | (n = 128) | (n = 211) | (n = 348) |
| Age of diagnosis (year) | 32 ± 6 | 32 ± 7 | 52 ± 8 | 57 ± 9 |
| Duration of disease (years) | 4 (0-31) | 2 (0-41) | 5 (0-24) | 4 (0-26) |
| Sex (M/F) (%) | 36/64 | 36/64 | 44/56 | 41/59 |
| Family history of diabetes | | | | |
| Father (%) | 45* | 0 | 21** | 0 |
| Mother(%) | 61 | 0 | 38 | 0 |
| Siblings(%) | 35 | 0 | 57 | 0 |
| At least 1 parent and 1 sibling(%) | 26 | 0 | 19 | 0 |
| Body mass index (kg/m²) | 25.7 ± 4.8 | 24.9 ± 4.4 | 24.4 ± 3.8 | 24.3 ± 3.8 |
| HbA_{lc} (%) | 7.3 (4.1-15.3) | 7.1 (3.8-16.8) | 7.7 (4.0-16.0) | 7.6 (4.2-19.7) |
| Fasting plasma glucose (nmol/l) | 7.4 (4.4-23.0) | 7.7 (2.8-21.4) | 7.8 (3.9-34.0) | 8.1 (3.0-24.5) |
| Fasting plasma C-peptide (nmol/l) | 0.47 (0.03-4.96) | 0.56 (0.09-1.62) | 0.57 (0.01-9.40) | 0.51 (0.01-8.22) |
| Insulin deficiency (%)† | 16 | 11 | 12 | 16 |
| Insulin treatment(%) | 11 | 13 | 9 | 11 |

Mean ± SD or median (range). Group 1: early onset (40 years) patients with a family history of diabetes; Group 2: early onset patients without a family history of diabetes; Group 3: late-onset patient (>40 years) with a family history of diabetes; Group 4: late-onset patients without a family history of diabetes.
†Insulin deficiency defined as fasting plasma C-peptide 0.2 nmol/l (Service, et al (1997) supra);
*P < 0.005 and
**P < 0.0001 for comparison between prevalence rates of paternal vs. maternal family histories.

TABLE 6
The clinical and biochemical characteristics of Chinese subjects carrying
the mt3243 mutation in the mitochondrial tRNA^{Leu} gene

| | Family A | Family B | Family C | Family D | Family E | | |
|---|---|---|---|---|---|---|---|
| | proband | proband | proband | proband | proband | II-2 | II-4 |
| Age of diagnosis (yr) | 32 | 22 | 31 | 70 | 33 | 38 | — |
| Duration of disease (yr) | 2 | 1 | 7 | 9 | 2 | 1 | — |
| Sex | F | M | F | M | M | F | F |
| Body mass index (kg/m²) | 18.2 | 22.4 | 18.6 | 25.3 | 27.6 | 21.6 | 19.6 |
| Waist-to-hip ratio | 0.79 | 0.81 | 0.75 | 0.90 | 0.86 | 0.79 | 0.75 |
| HbA_{1c} (%) | 5 | 7.7 | 6.8 | 5.9 | 11.3 | 7.5 | 4.3 |
| Fasting glucose(mmol/l) | 5.3 | 11.6 | 9.8 | 5.8 | 15.7 | 7.1 | 4.4 |
| Fasting C-peptide (nmol/l) | 0.43 | 0.23 | 0.30 | 0.7 | 0.72 | 0.83 | 0.4 |
| Fasting insulin (mIU/I) | 13.4 | 26.0 | ND | ND | 20.6 | 16.8 | 13.2 |
| Insulin resistance* | 3.2 | 13.4 | 5.5 | ND | 14.4 | 5.3 | 2.6 |
| Treatment | Oral drugs | Oral drugs | insulin | Oral drugs | Oral drugs | Oral drugs | — |
| Audiogram | Normal | Normal | High tone | High tone | Normal | ND | ND |
| | | | impairment | Impairment | | | |
| Mt3243 level (%) | 13 | 1 | 14 | 1 | 5 | 9 | 4 |

*HOMA method,
ND, not determined

Family D

The 79-year-old proband was diagnosed with diabetes at the age of 70 years. An audiometry test revealed high tone sensorineural impairment. Neither of the parents and nor any siblings were available for screening or known to have diabetes.

Family E

The 35-year-old proband (II-3) was diagnosed with diabetes at the age of 33 years and was treated with diet. One of the sisters (II-4) had normal glucose tolerance while two sisters (II-1 and II-2) were diagnosed to have diabetes at the age of 30 and 38 years, respectively. The father (I-1) and mother (I-2) also had diabetes at the age of 50 and 35 years, respectively. All the diabetic and nondiabetic sisters who came for screening had the mt3243 mutation. One of the diabetic sisters (II-1) had high tone sensorineural impairment whereas the audiogram of the proband was normal.

### EXAMPLE 3

### The Role of the Amylin Gene S20G Mutation in Early Onset Type 2 Diabetes and in the Regulation of Cholesterol Metabolism in Chinese

This example illustrates the distribution of the amylin gene S20G mutation in Hong Kong Chinese with or without Type 2 diabetes, and its influences on β-cell function and metabolic profiles. The data are consistent with the conclusion that the S20G mutation in the amylin gene may contribute to early occurrence of Type 2 diabetes, and that it may also influence lipid metabolism in the Chinese population.

Experimental Design and Methods

Subjects

The study protocol was approved by the Clinical Research Ethics Committee of the Chinese University of Hong Kong. Informed consent was obtained from each of the participants. For the study, 227 early-and 235 late-onset Type 2 diabetic patients (defined as age at diagnosis ≦40 and >40 years respectively), as well as 126 non-diabetic subjects (defined as fasting plasma glucose <6 mmol/1), were consecutively recruited at the Diabetes Centre of the Prince of Wales Hospital. Type 2 diabetes was asscertained according to the World Health Organisation criteria (Anonymous (1997) Diabetes Care 20: 1183). None of the patients had typical presentations of Type 1 diabetes, such as acute symptoms and heavy ketonuria (>3+), history of diabetic ketoacidosis or continuous need for insulin treatment within I year of diagnosis. Patients who had anti-glutamic acid decarboxylase autoantibody (Ko, et al (1998) Ann Clin Biochem 35: 761) diabetes-causing mutations in the glucokinase and hepatonuclear factor-1α and 4-α genes (Ng et al (1999) Diabetic Medicine 16: 956) were excluded from the study.

Clinical and Biochemical Measurements

Patients fasted at least 8 hours prior to their clinical examinations. Blood pressures were taken, after they remained sitting for at least for 5 min using a standard mercury sphygmomanometer. Body height and weight, and waist and hip circumferences were taken while the patients were standing in light clothing but wearing no shoes. Measurements of fasting plasma glucose and lipids were performed by routine assays in the Department of Chemical Pathology at the Prince of Wales Hospital. Levels of total cholesterol and triglyceride were assayed enzymatically with commercial reagents(Centrichem, chemistry system, Baker Instrument Co., Allentown, Pa.). HDL-cholesterol was determined after fractional precipitation with dextran sulfate-MgCl₂ and LDL-cholesterol, calculated by the Friedewald's equation (Friedewald et al (1972) Clin Chem 18:499). HbA_{1c} was measured using an automated ion-exchange chromatographic method (BioRad, Hercules, Calif., USA; normal range: 5.1-6.4%). Plasma levels of C-peptide were measured by radioimmunoassays using commercial kits (#7350104 and #141 respectively, Novo Nordisk, Denmark). The detection range was from 0.01 to 1.0 pmol/1. Insulin deficiency was defined as fasting plasma C-peptide level <0.2 pmol/1 (Service et al (1997) Diabetes Care 21: 987).

Mutation Detection

The S20G mutation creates a MSP I restriction fragment length polymorphism (RFLP), which can be detected using PCR-RFLP analysis (Sakagashira et al (1996) Diabetes 45: 1279). Briefly, DNA fragments spanning the mutation site were amplified by PCR using the primers 5′-TCACATTTGTTCCATGTTAC-3′ (SEQ ID NO: 30) and 5′-CAATAACTATAGAGTTACATTG-3′ (SEQ ID NO: 31), at the annealing temperature of 56° C. Each of the PCR products was then digested overnight with 5 units of the restriction enzyme MSP I (#R6401, Promega, Wis., USA) at 37° C. Alleles were separated on 2.5% agarose gel. The wild-type allele showed a 239 bp product whereas the mutant allele showed 99 and 140 bp products.

Statistical Analysis

Continuous variants were expressed as mean ±SD. Chi-square test was used for the analysis of proportions. Differences between continuous variables were analysed by the student's t-test using the statistical package for social sciences (SPSS Inc., Chicago, USA). ap value <0.05 was considered to be statistically significant.

Results

Table 7 summaries the demographic data of the subjects involved in the study.6 early-and 1 late-onset patients heterozygous for the amylin S20G mutation (2.6% vs 0.4%, p=0.055) were identified. None of the non-diabetic subjects had the S20G mutation (Table 7).

In the early-onset group, 5 out of the 6 mutation-carrying patients had satisfactory glycemic control with diet and/or oral drug medications, and had fasting plasma C-peptide concentrations of greater than 0.2 pmol/1 (Table 8). Moreover, the mutation carriers had lower total cholesterol (4.3±0.9 vs 5.3 1.1, p=0.02) and LDL-cholesterol (2.3±0.7 vs 3.2±0.9, p=0. 01) (Table 9) than those without the mutation. The patients with or without the S20G mutation were of a comparable age (34±6 vs 35±8, p>0.05).

TABLE 7
Clinical characteristics of the early and late-onset patients as
well as non-diabetic subjects, and the distribution
of the amylin gene S20G mutation.

| | Type 2 diabetes | | |
|---|---|---|---|
| | Control | | |
| | subjects | Early-onset | Late-onset |
| Clinical characteristics | | | |
| n | 126 | 227 | 235 |
| Age (years) | 34.9 ± 10.4 | 36.8 ± 6.7 | 59.4 ± 10.1 |
| Sex ratio (M/F) | 1:1.75 | 1:1.97 | 1:1.33 |
| Age of diagnosis (years) | NA | 31.7 ± 4.6 | 54.3 ± 9.8 |
| Body mass index (kg/m2) | 22.3 ± 3.4 | 25.1 ± 4.5 | 24.2 ± 3.6 |
| Waist to hip ratio | 0.77 ± 0.05 | 0.85 ± 0.07 | 0.89 ± 0.06 |
| Systolic blood | 114 ± 10 | 119 ± 17 | 137 ± 21 |
| pressure (mmHg) | | | |
| Diastolic blood | 64 ± 9 | 76 ± 10 | 82.0 ± 11 |
| pressure (mmHg) | | | |
| HbA_{lc} (%) | — | 7.6 ± 2.0 | 8.1 ± 2.2 |
| Total cholesterol (mmol/l) | 4.7 ± 0.9 | 5.3 ± 1.2 | 5.6 ± 1.2 |
| HDL-cholesterol (mmol/l) | 1.4 ± 0.3 | 1.3 ± 0.4 | 1.3 ± 0.4 |
| LDL-cholesterol (mmol/l) | 2.9 ± 0.8 | 3.2 ± 0.9 | 3.6 ± 1.1 |
| Triglyceride (mmol/l) | 0.9 ± 0.5 | 1.7 ± 1.8 | 1.8 ± 1.5 |
| Genotypes | | | |
| Wild-type allele | 126 | 221 | 234 |
| homozygotes | | | |
| Heterozygotes | 0 | 6 | 1 |
| Mutant allele | 0 | 0 | 0 |
| homozygote | | | |
| S20G allele | 0 | 2.6^{‡} | 0.4 |
| frequency (%) | | | |

Mean ± SD;
‡p = 0.055

TABLE 8
Fasting plasma levels of glucose, HbA1c and
C-peptide in early-onset Type 2 diabetic patients
with an amylin gene S20G mutation

| | | | | C- | | |
|---|---|---|---|---|---|---|
| | Onset | Duration | | peptide | HbA_{1c} | Glucose |
| Patient | age | (years) | Treatment | (pmol/l) | (%) | (mmol/l) |
| Index a | 29 | 1 | Diet | >1.0 | 6.2 | 5.3 |
| Index b | 25 | 18 | Oral drugs | >1.0 | 7.2 | 7.2 |
| Index c | 35 | 3 | Diet | >1.0 | 8.2 | 9.3 |
| Index d | 28 | 2 | Diet + oral | 0.02 | 5.4 | 4.9 |
| Index e | 36 | 1 | Diet + oral | 0.51 | 5.8 | 14.0 |
| Index f | 13 | 13 | Insulin | — | 11.5 | 7.2 |

TABLE 9
Comparisons of clinical characteristics and biochemical
measurements between early-onset S20G mutation-carrying
patients and early-onset patients without the S20G mutation.

| Patients | Age | Sex | BMI | WHR | SBP | DBP | HbA_{1c} | Triglyceride | Total-C | HDL-C | LDL-C |
|---|---|---|---|---|---|---|---|---|---|---|---|
| Index a | 30 | F | 23.2 | 0.76 | 114 | 79 | 6.2 | 0.76 | 3.8 | 1.04 | 2.5 |
| Index b | 43 | F | 17.9 | 0.78 | 138 | 74 | 7.2 | 0.46 | 5.8 | 2.21 | 3.3 |
| Index c | 38 | M | 28.2 | 0.88 | 128 | 90 | 8.2 | 2.90 | 4.7 | 1.06 | 2.3 |
| Index d | 30 | F | 23.2 | 0.75 | 105 | 66 | 5.4 | 0.99 | 4.0 | 1.37 | 2.2 |
| Index e | 37 | F | 24.0 | 0.89 | 126 | 80 | 5.8 | 2.76 | 3.3 | 0.87 | 1.2 |
| Index f | 26 | M | 29.1 | 0.89 | 120 | 81 | 11.5 | 0.59 | 4.2 | 1.71 | 2.2 |
| Mutation + (n = 6) | 34 ± 6 | — | 24 ± 4 | 0.8 ± 0.1 | 122 ± 16 | 78 ± 8 | 7.4 ± 2.0 | 1.4 ± 1.1 | 4.3 ± 0.9 | 1.3 ± 0.5 | 2.3 ± 0.7 |
| Mutation − (n = 221 | 35 ± 8 | — | 25 ± 4 | 0.9 ± 0.1 | 119 ± 17 | 76 ± 10 | 7.6 ± 2.3 | 1.7 ± 2.5 | 5.3 ± 1.1* | 1.3 ± 0.4 | 3.2 ± 0.9** |

Mean ± SD;
*p = 0.02;
**p = 0.01
BMI, body mass index (kg/m²);
WHR, waist to hip ratio;
SBP, systolic blood pressure (mmHg);
DBP, diastolic blood pressure (mmHg);
Total-C, total cholesterol (mmol/l);
HDL-C, HDL-cholesterol (mmol/l);
LDL-cholesterol (mmol/l).

The genetic association between the S20G mutation and early-onset Type 2 diabetes (Table 7 ; Sakagashira et al (1996) supra) is consistent with the physiological data that amylin may play a role in the pathogenesis of the disease (Cooper (1994) Proc Natl Acad Sci 84: 8628). Early onset of Type 2 diabetes is in fact not uncommon (Rosenbloom et al (1999) Diabetes Care 22: 345), although type 2 diabetes is classically a late-onset disease. However, early-onset patients appear to be heterogenous in etiology. In Hong Kong Chinese, in particular, maturity-onset Type 2 diabetes of the young as well as atypical autoimmune diabetes are present, but accounting for only a small proportion of the overall early-onset population (Ng et al (1999) supra; Ko et al (1998) supra). The S20G mutation may also explain some of the early-onset cases.

The S20G mutation carriers usually did not require insulin for glycemic control, and did not appear to be insulin deficient (Table 8). These findings are different from previous observations that the S20G mutation may be associated with poor glycemic control as well as β cell dysfunction (Sakagashira et al (1996) supra; Chuang et al (1998) supra). The reported Japanese S20G carriers (Sakagashira et al (1996) supra) had an average diabetes duration of approximately 20 years at the time they were tested. That they commonly required insulin treatment may be due to the deterioration in glycemic control during their long diabetes course, not necessary the presence of the mutation.

Moreover, the mutation appears to be associated with lower plasma levels of total cholesterol and LDL-cholesterol (Table 9). This is in keeping with the recent finding that pramlintide (a synthetic human amylin analog) was able to lowers plasma levels of total cholesterol and LDL-cholesterol in Type 2 diabetic patients (Thompson et al (1998) Diabetes Care 21: 987). Few studies to date have been focused on the relationships between amylin action and lipid profiles These data and those from Thompson and co-workers are consistent with the conclusion that amylin may also play a role in the regulation of cholesterol metabolism.

### EXAMPLE 4

### The Significant Roles of Genetics and Obesity in Familial Early-Onset Type 2 Diabetes in Chinese Patients

This example illustrates the prevalence of known molecular defects in separate cohorts of Chinese patients with early-and late-onset familial Type 2 diabetes. The genes studied are those that have been found to be associated with diabetes and which may contribute to early onset of the disease under gene-gene and gene-environmental influences, including glucokinase (MODY2), HNF-1α (MODY3), and the A3243G mutation in the mitochondrial DNA coding for tRNA^{Leu(UUR)} (mt3243) that has been associated with a form of diabetes characterized by maternal inheritance and deafness (van den Ouweland, et al (1992) Nature Genet. 1: 368).

Experimental Design and Methods

Subjects

The Prince of Wales Hospital (PWH) is a regional teaching hospital in Hong Kong. Its catchment area has a population of 1.2 million, accounting for 20% of the total population in Hong Kong. There is a lack of long term health care programs in Hong Kong, and medical insurance is not widely available. Many patients with chronic diseases such as diabetes are managed in public hospitals or clinics where they pay only a nominal fee. Hence, except for high social classes, the patients are largely representative of the diabetic population in Hong Kong. Since 1995, all patients attending the diabetes clinic of the PWH have been entered into the PWH Diabetes Registry after undergoing a structured assessment (Piwemetz, et al (1993) Diabetic Med 10:371; Chan, et al (1997) Hosp Auth Qual Bull 2:3). During the study period, a separate cohort of 150 young patients with early-onset diabetes (age ≦40 years and age at diagnosis ≦35 years) who underwent the structured assessment were recruited consecutively from the diabetes clinics at the PWH to form the Young Chinese Diabetes Database (Ko, et al (1998) Ann Clin Biochem 35: 761). The 150 cases in the Young Chinese Diabetes Database, 92 and 53 patients, respectively, were selected for the present study as they satisfied the following criteria: All these 145 young patients had early-onset (current age and age at diagnosis ≦40 years) Type 2 diabetes (1985 WHO criteria, Geneva) and a positive family history for diabetes (at least 1 first degree relative with diabetes). Patients with classical Type 1 diabetes (acute ketotic presentation or continuous requirement of insulin within 1 year of diagnosis) were excluded from the study.

The prevalence of anti-GAD (Ko, et al (1998) supra), mt3243 (Smith, et al (1997) Diabetic Med 14: 1026; Ng, et al (2000) 52: 557) and amylin gene mutations (Lee, et al (2001) J. Endocrinol) amongst patients from the Young Chinese Diabetes Database has been reported. Additionally, the prevalence of mt3243 (Ng, et al (2000) supra), amylin (Lee, et al (2001) supra), glucokinase, HNF-1α and HNF-4α gene mutations (Ng, et al(1999) Diabetic Med 16:956) in a separate cohort from the PWH Diabetes Registry has been reported. In this study, screening for glucokinase and HNF-1α gene mutations was extended to the 53 patients from the Young Chinese Diabetes Database. The HNF-4α gene was not screened in this cohort due to the expected low frequency of mutations. (None were found in the 92 patients from the PWH Diabetes Registry (Ng, et al (1999) supra)). Screening for anti-GAD was extended to the 92 patients from the PWH Diabetes Registry.

Nineteen (13%) of these 145 young patients with familial diabetes met the minimal criteria for MODY (age at diagnosis ≦25 years and presence of diabetes in two consecutive generations). Altogether 10 out of 20 families with probands carrying putative diabetogenic gene mutations were recruited for a 75-gram OGTT and clinical assessment. The 1999 WHO classification was used to define the glycemic status of the family members (WHO, Geneva, 1999). For comparison of clinical characteristics of the early-onset patients, 290 sex-matched patients with late-onset diabetes (age at diagnosis >40 years) and a positive family history of diabetes were randomly selected from the current 1800 cases in the PWH Diabetes Registry. One hundred healthy Chinese (age 33±10 years, 40 males and 60 females) were selected as control subjects from hospital staff and students for screening for the gene variants identified in the study patients. Informed consent was obtained from each subject for a blood sample to be taken for DNA extraction and measurement of biochemical indices. This study was approved by the Clinical Research Ethics Committee of The Chinese University of Hong Kong.

Clinical Studies

All patients underwent a structured assessment based on the Europe DiabCare Protocol. They had documentation of their family history of diabetes, age at diagnosis and anthropometric indices (Piwemetz, et al (1993) supra; Chan, et al (1997) supra). Body mass index (BMI) was used as an index of general obesity. Waist circumference, which is highly correlated in Chinese with visceral fat accumulation measured by magnetic resonance imaging (Anderson, et al (1997) Diabetes Care 20: 1854), was used as an index of central obesity. After an overnight fast, venous blood was sampled for measurement of plasma glucose, insulin, HbA_{1c}, total cholesterol (TC), HDL-C, LDL-C (calculated), triglyceride (TG) and anti-GAD. A morning spot urine sample was collected for assessment of albuminuria. Retinopathy and sensory neuropathy were assessed as previously described (Ko, et al (1999) J Diabetes Complications 13: 300).

General obesity was defined as a BMI≧25 kg/m² using the recent Asian criteria (WHO, Western Pacific Region, 2000). Albuminuria was defined as an albumin: creatinine ratio (ACR)≧3.5 mg/mmol in a spot urine sample (Schwab, et al (1992) Diabetes Care 15: 1581). The HOMA IR index (fasting plasma insulin×glucose/22.5) derived from the HOMA equation was used to assess insulin resistance (Matthews, et al (1985) Diabetologia 28: 412).

Biochemical Assays

Plasma glucose, HbA_{1c}, lipids, urinary albumin and creatinine were measured by routine assays in the Department of Chemical Pathology at the PWH (see Chan, et al (1996) Diabetic Med 13:150). Plasma insulin was measured in non-insulin treated patients by a radioimmunoassay (Pharmacia, Sweden) with intra-and inter-assay CVs of 6% and 13.8%, respectively. Anti-GAD was measured by a radioimmunoprecipitation assay (Chen, et al (1993) Pediatr Res 34: 785). The upper normal limit of 18 units, is applicable to Asian and European subjects (Tuomi, et al (1995) Clin Immunol Immunopath 74: 202, Chen, et al (1993) supra).

Genetic Analysis

The minimal promoter regions and exons of the glucokinase (β-cell form), HNF-1α and HNF-4α (HNF-4α 2 form) genes were screened for mutations by direct sequencing of PCR products (see Froguel, et al (1993) N Engl J Med 328: 697; Yamagata, et al (1996) Nature 384: 455; Yamagata, et al (1996) Nature 384: 458). One previously unreported mutation in HNF-1α (A116V) and two previously unreported mutations in glucokinase (V101M and Q239R) were identified in this study. HNF-1α A116V was screened by using the forward primer 5′-CATGCACAGTCCCCACCCTCA-3′ (SEQ ID NO:34) and reverse primer 5′-TCCCACTG ACTTCCTTTCC-3′ (SEQ ID NO:35) for PCR amplification followed by digestion with HphI. The wild-type allele showed 44 and 397 bp products whereas the mutant allele showed 44, 136 and 261 bp products. Glucokinase V101M was screened by using the forward primer 5′-GTCCCTGA-GGCTGACACACTT-3′ (SEQ ID NO: 24) and reverse primer 5′-AGCTGGGCCCTGAGATCC-TGCA-3′ (SEQ ID NO: 25) for PCR amplification followed by digestion with Hsp9211. The wild-type allele showed 20, 56 and 174 bp products whereas the mutant allele showed 20,42,56, and 132 bp products. Glucokinase Q239R was screened by using the forward primer 5′-AGGAACC-AGGCCCTACTCCG-3′ (SEQ ID NO: 36) and reverse primer 5′-TACTCCAGCAGGAACTC-GTCC-3′ (SEQ ID NO: 37) for PCR amplification followed by digestion with Acil. The wild-type allele showed 70 and 134 bp products whereas the mutant allele showed 33,70 and 101 bp products. The occurrence of putative mutations in family members of the probands (Table 10) and control subjects was determined by PCR-RFLP. Mt3243A→G and amylin gene S20G mutations were determined by PCR-RFLP as described (Sakagashira, et al (1996) Diabetes 45: 1279; Smith, et al (1997) supra).

Statistical Analysis

Normally distributed data are expressed as mean ± SD. Data with skewed distributions were normalised by logarithmic transformation. The resultant means were antilogarithmically transformed and expressed as geometric mean together with 25 and 75 percentiles. Chi square test and Student's unpaired t tests were used for between-group comparisons. A p value<0.05 (2-tailed) was considered to be significant. All statistical analyses were performed using the Statistical Package for Social Sciences (SPSS for Windows, version 9.0).

Results

Prevalence of Putative Gene Mutations and Anti-Gad in Patients with Familial Early-Onset Diabetes

Amongst the 145 patients with familial early-onset diabetes, there were 20 (14%) with putative mutations, involving the HNF-1α gene, 7 (5%), glucokinase gene, 6 (4%), mt3243,4 (3%), and amylin S20G, 3 (2%). Anti-GAD was positive in 6 (4%). No mutation in the HNF-4α gene was found in the 92 patients from the PWH Diabetes Registry. All mutations identified in the HNF-1α and glucokinase genes were previously unreported (Table 10). The HNF-1α G20R and glucokinase Q239R mutations were found in 4 unrelated patients. None of these mutations were found in 100 healthy control subjects.

Family Cosegregation Study of Gene Mutations

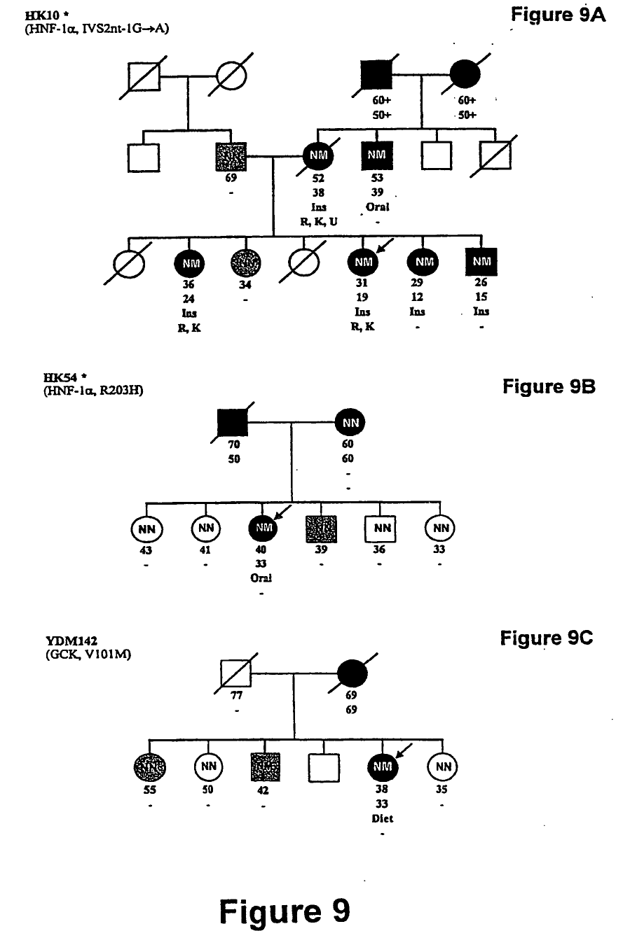

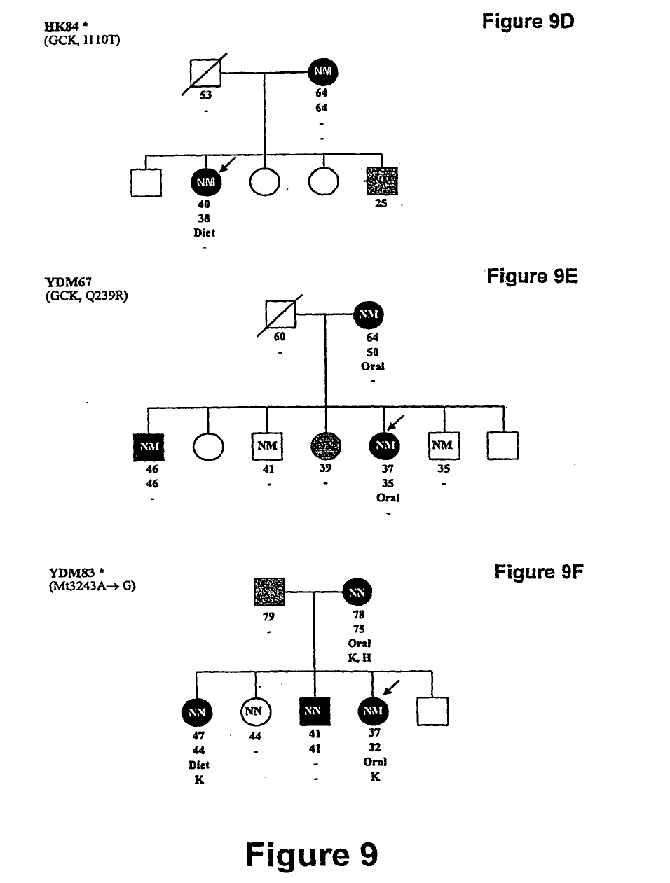

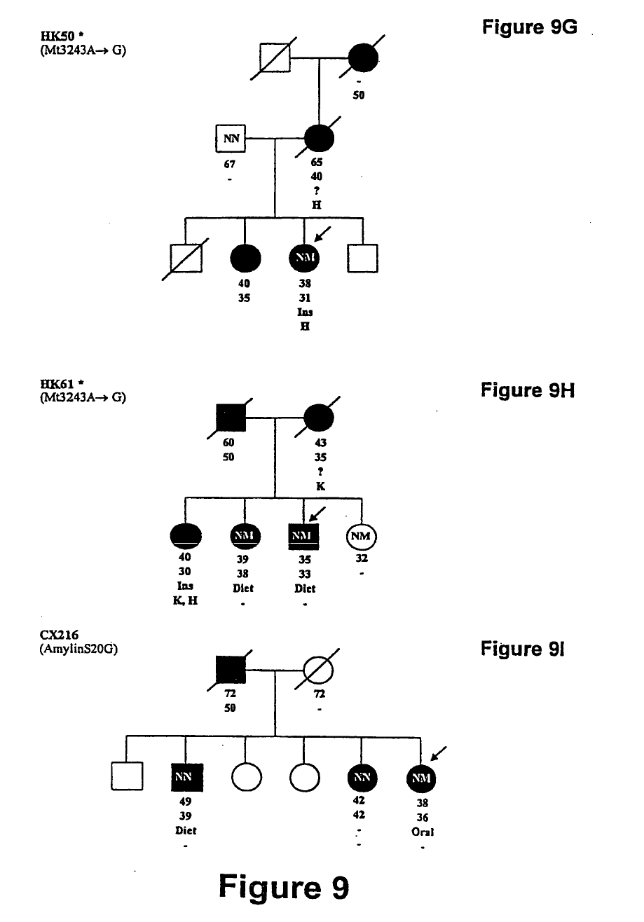

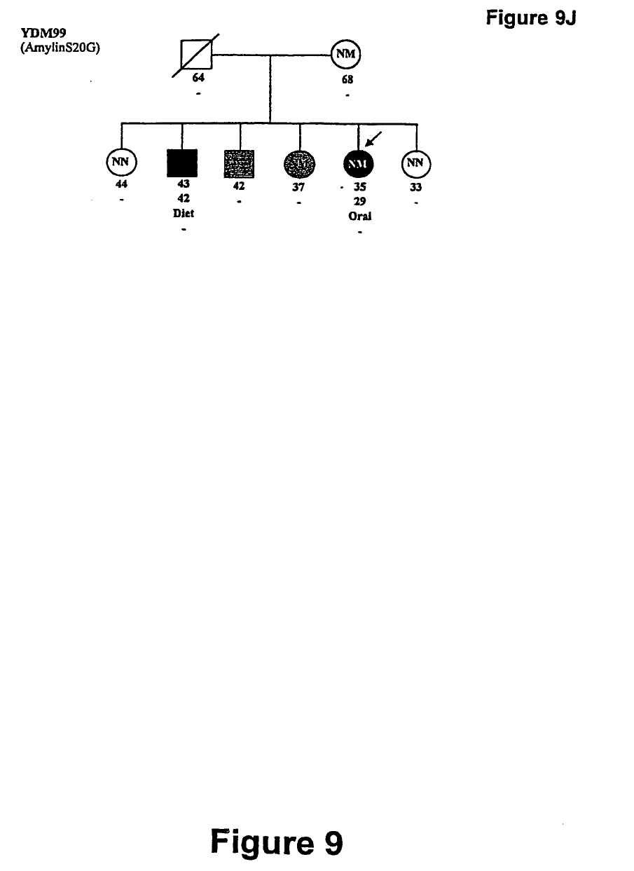

Amongst the 20 patients carrying putative gene mutations, 10 families were recruited for cosegregation study (FIG. 9). Cosegregation of a mutation with clinical diabetes or glucose intolerance were observed in 4 families: HK10 with HNF-1α IVS2nt-1G→A, YDM142 with glucokinase V101M, HK84 with glucokinase I110T and HK50 with mt3243. Segregation was inconclusive in the other 6 families. For the families HK54 with HNF-1α R203H, YDM83 with mt3243 and CX216 with amylin S20G mutations, only the probands, and none of the diabetic or non-diabetic family members who presented for screening, carried the gene mutations. For YDM67 with glucokinase Q239R, HK61 with mt3243 and YDM99 with amylin S20G mutations, the mutations were found in both diabetic and non-diabetic family members. Amongst the 3 families with glucokinase mutations, all mutation carriers from the families YDM142 and HK84 had higher fasting plasma glucose concentrations (5.8-8.9 mmol/1) than those with no mutation (4.2-5.3 mmol/1). On the other hand, the 4 mutation-carrying siblings of proband YDM67 had a normal fasting plasma glucose concentration (4.0-5.6 mmol/1) irrespective of their glycemic status.

TABLE 10
Mutations in the HNF-1 α and glucokinase genes in Chinese subjects
with early-onset diabetes mellitus

| Subject | Location | Codon/nt | Nucleotide change | Designation |
|---|---|---|---|---|
| HNF-1 _{α}mutation | | | | |
| HK90*, YDM42† | Exon 1 | 20 | GGG (GLy)→AGG (Arg) | G20R |
| YDM20† | Exon 2 | 116 | GCG (Ala)→GTG(Val) | A116V |
| HK10* | Intron2/Exon3 | nt-1 | AG→AA at splice acceptor site | IVS2nt-1G→A |
| HK54* | Exon 3 | 203 | CGT (Arg)→CAT (His) | R203H |
| HK30* | Exon 6 | 432 | TCC (Ser) →TGC (Cys) | S432C |
| HK92* | Exon 10 | 618 | ATC (Ile)→ATG (Met) | 1618M |
| Glucokinase mutation | | | | |
| YDM142† | Exon 3 | 101 | GTG (Val)→ATG (Met) | V101M |
| HK84* | Exon 3 | 110 | ATC (Ile)→ACC (Thr) | I110T |
| HK38* | Exon 3 | 119 | GCT (Ala)→GAT (Asp) | A119D |
| YDM67†, YDM144† | Exon 7 | 239 | CAG (Gln)→CGG (Arg) | Q239R |
| HK15* | Exon 9 | 385 | GGG (Gly)→GTG(Val) | G385V |

*reported in previous studies (Ng, et al (1999) Diabetic Med 16: 956; Ng, et al (2000) Diabetologia 43: 816)
†newly found in the present study

Clinical Characteristics of Patients with Familial Early-Onset Diabetes of Unknown Cause Compared with Familial Late-Onset Diabetes

Although 26 of the patients with early-onset diabetes carried putative gene mutations associated with diabetes or the autoimmune indicator, anti-GAD antibodies, the causes of diabetes in the other 119 patientsremain to be determined. These young patients with diabetes of unknown cause (age at diagnosis 30±6 years) differed clinically from the 290 late-onset patients (age at diagnosis 52±8 years) (Table 11). Thus, despite a positive family history of diabetes in all patients in both groups, those with early-onset diabetes more frequently had a father with diabetes (39% vs. 22%) and a mother with diabetes (63% vs. 41%), but less frequently a sibling with diabetes (30% vs. 53%) (p<0.001). The early-onset patients had a higher BMI but lower BP and increased prevalence of retinopathy and neuropathy as compared to the late-onset patients. The early-onset patients had better glycemic control (glucose and HbA_{1c}) as well as higher fasting insulin concentrations than the late-onset patients. Notwithstanding similar mean disease duration of only 4 years, both the early-and late-onset patients had a disproportionately high prevalence of albuminuria, 40% and 38%, respectively, as compared with the prevalence rates of other microangiopathic complications. Insulin resistance, as assessed by the HOMA IR index, was similar between the two groups of non-insulin treated patients. The proportion of patients treated with insulin was similar in both groups (8% vs. 7%) but fewer patients with early-onset diabetes were treated with oral drugs (33% vs. 61%, p<0.001) as compared to the late-onset group.

Clinical Characteristics of the Patients with Familial Early-Onset Diabetes of Unknown cause and Familial Late-Onset Diabetes Classified According to Obesity Index

Due to the high prevalence of general obesity in both early-onset patients of unknown cause and late-onset patients (55% and 46%, respectively), the association of obesity with cardiovascular risk factors and complications in these patients was further analyzed (Table 11). Amongst the early-onset patients, the obese patients had worse glycemic control (HbA_{1c}) as well as a higher systolic BP, a more adverse lipid profile (higher TG, lower HDL-C and higher TC/HDL-C), and higher fasting insulin than the non-obese patients. They were also more insulin resistant (HOMA IR index) and had a higher prevalence of retinopathy and albuminuria than the non-obese patients. Amongst the late-onset patients, the obese patients had better glycemic control (glucose and HbA_{1c}) than the non-obese patients. However, they had a higher systolic and diastolic BP, and a higher fasting insulin than the non-obese patients. The degree of insulin resistance and prevalence of complications were similar in the two groups.

TABLE 11
Comparison of clinical features of Chinese patients with familial Type 2 diabetes
according to age of diagnosis of diabetes and obesity status

| | | | Early-onset | | Late-onset | |
|---|---|---|---|---|---|---|
| | Early-onset patients | Late-onset | non-obese | Early-onset | non-obese | Late-onset |
| | with unknown etiology | patients | patients | obese patients | patients | obese patients |
| N | 119 | 290 | 54 | 65 | 156 | 134 |
| Sex | | | | | | |
| Male (%) | 37 (31) | 98 (34) | 12 (22) | 25 (38) | 56 (36) | 42 (31) |
| Female (%) | 82 (69) | 192 (66) | 42 (78) | 40 (62) | 100 (64) | 92 (69) |
| Current age (yr) | 34 ± 5 | 56 ± 9‡ | 34 ± 5 | 33 ± 5 | 56 ± 10 | 55 ± 9 |
| Age at diagnosis (yr) | 30 ± 6 | 52 ± 8‡ | 31 ± 5 | 29 ± 6 | 52 ± 8 | 52 ± 8 |
| Duration of disease (yr) | 4.0 ± 3.9 | 4.0 ± 4.2 | 3.9 ± 3.8 | 4.2 ± 4.0 | 4.5 ± 4.4 | 3.5 ± 3.9† |
| Family history | | | | | | |
| Father | 46 (39) | 64 (22)‡ | 21 (39) | 27 (42) | 28 (18) | 36 (27) |
| Mother | 75 (63) | 119 (41)‡ | 38 (70) | 39 (60) | 65 (42) | 54 (40) |
| Sibling | 36 (30) | 154 (53)‡ | 15 (28) | 20 (31) | 85 (54) | 69 (51) |
| BMI (kg/m²) | 26.2 ± 4.7 | 25.0 ± 3.7† | 22.3 ˜ 1.8 | 29.5 ± 3.8‡ | 22.4 ± 1.8 | 28.0 ± 3.1‡ |
| Waist circumference (cm) | | | | | | |
| Male | 90 ± 11 | 87 ± 9 | 78 ± 6 | 95 ± 9‡ | 81 ± 6 | 94 ± 7‡ |
| Female | 81 ± 11 | 83 ± 9 | 74 ± 5 | 89 ± 10‡ | 78 ± 6 | 89 ± 8‡ |
| Systolic BP (mmHg) | 117 ± 14 | 136 ± 22‡ | 114 ± 13 | 120 ± 14† | 134 ± 23 | 139 ˜ 21† |
| Diastolic BP (mmHg) | 75 ± 9 | 83 ± 11‡ | 74 ± 9 | 77 ± 10 | 80 ± 11 | 86 ± 12‡ |
| Triglyceride (mmol/l) | 1.4 (0.9-2.0) | 1.4 (1.0-2.0) | 1.0 (0.7-1.5) | 1.7 (1.0-2.4)‡ | 1.4 (0.9-1.9) | 1.6 (1.1-2.1) |
| Total cholesterol (mmol/l) | 5.3 ± 1.2 | 5.6 ± 1.3 | 5.1 ± 1.0 | 5.4 ± 1.4 | 5.6 ± 1.3 | 5.5 ± 1.2 |
| HDL-C (mmol/l) | 1.2 ± 0.3 | 1.2 ± 0.3 | 1.3 ± 0.3 | 1.1 ± 0.3‡ | 1.3 ± 0.4 | 1.2 ± 0.3 |
| TC/HDL-C | 4.7 ± 1.8 | 4.7 ± 1.5 | 4.0 ± 1.0 | 5.3 ± 2.2‡ | 4.7 ± 1.7 | 4.7 ± 1.3 |
| LDL-C (mmol/l) | 3.3 ± 0.9 | 3.5 ± 1.0 | 3.2 ± 0.8 | 3.4 ± 1.0 | 3.5 ± 1.0 | 3.5 ± 1.0 |
| Fasting glucose (mmol/l) | 8.2 ± 3.1 | 9.1 ± 3.6† | 7.6 ± 2.8 | 8.7 ± 3.3 | 9.6 ± 3.8 | 8.6 ± 3.3† |
| HbA_{1c} (%) | 7.5 ± 1.8 | 8.0 ± 1.9† | 7.1 ± 1.8 | 7.9 ± 1.8† | 8.2 ± 2.1 | 7.7 ± 1.5† |
| Fasting insulin (pmol/l)* | 105 (72-164) | 87 (51-146)† | 89 (60-149) | 120 (78-179)† | 76 (43-129) | 99 (57-157)† |
| HOMA IR index* | 34.7 (22.9-55.8) | 33.1 (19.2-62.8) | 27.5 (16.0-46.7) | 42.7 (27.6-58.2)† | 29.9 (14.7-61.9) | 36.4 (19.7-64.2) |
| Urinary albumin creatinine | 2.6 (0.7-6.1) | 2.8 (0.8-7.1) | 1.2 (0.6-1.9) | 5.1 (0.9-24.1)‡ | 2.6 (0.9-5.8) | 3.2 (0.8-8.4) |
| ratio (mg/mmol) | | | | | | |
| Treatment (%) | | | | | | |
| Diet | 71 (60) | 93 (32) | 35 (65) | 36 (55) | 55 (35) | 38 (28) |
| Oral drugs | 39 (33) | 176 (61)‡ | 17 (31) | 22 (34) | 89 (57) | 87 (65) |
| Insulin | 9 (8) | 21 (7) | 2 (4) | 7 (11) | 12 (8) | 9 (7) |
| Retinopathy (%) | 10 (8) | 62 (21)† | 1 (2) | 9 (14)† | 38 (24) | 24 (18) |
| Albuminuria (%) | 48 (40) | 110 (38) | 8 (15) | 40 (62)‡ | 53 (34) | 57 (43) |
| Neuropathy (%) | 4 (3) | 29 (10)† | 2 (4) | 2 (3) | 13 (8) | 16 (12) |

Data are compared between early- and late-onset patients, between early-onset non-obese and obese patients, and between late-onset non-obese and obese patients
Data are expressed as n (%), mean ± SD or geometric mean (25 and 75 percentiles)
*only measured in patients not treated with insulin
†p < 0.05
‡p < 0.001

### EXAMPLE 5

### An Illustration of a Chinese Family with Hepatocyte Nuclear Factor-1α Diabetes (MODY3) that Emphasizes the Need for Early Diagnosis and Appropriate Treatment

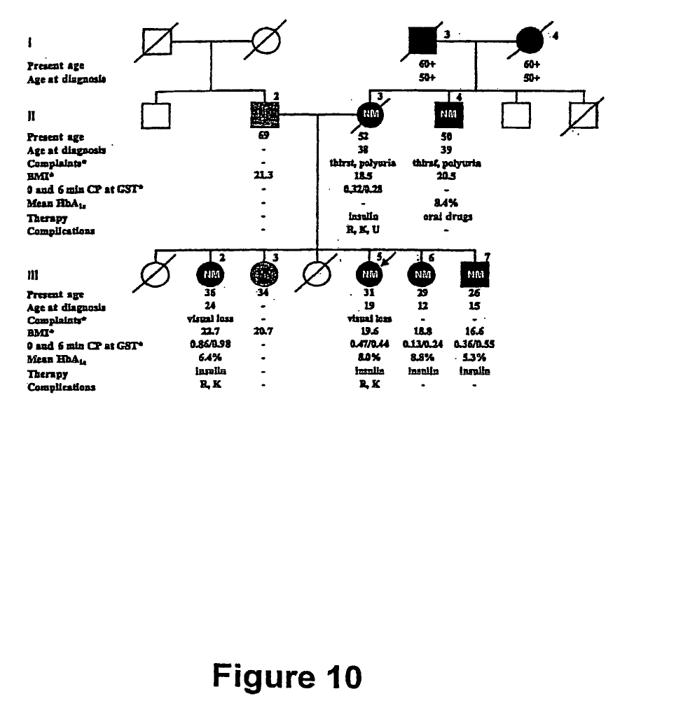

This example reports the clinical course of HNF-1α diabetes/MODY 3 in a Chinese family with early-onset diabetes and severe complications (FIG. 10) (Chan, et al (1990) Diabetic Medicine 7: 211). This family highlights the importance of early diagnosis and prompt treatment in the improvement of clinical outcome even in genetically susceptible subjects.

Three family members in the proband's family had severe diabetic complications when they were referred for treatment. The proband (III-5), 19 years of age, had severe proliferative retinopathy, heavy proteinuria (1.4 g protein a day) and necrobiosis lipoidica. She had been diagnosed with Type 2 (non-insulin-dependent) diabetes mellitus 3 months earlier and was treated with glibenclamide. Retinal photocoagulation treatment was initiated and she was started on insulin and an ACE inhibitor. She subsequently developed hypertension and progressed to end-stage renal disease requiring dialysis by the age of 30 years. Her mean HbA_{1c} was 8.0% over the years. She is currently receiving 42 units of insulin.

Her older sister (III-2) had a vitreous haemorrhage and had been treated with insulin since diagnosis at the age of 24 years. She became blind and had nephropathy (0.8 g protein a day) 2 years later. She is currently treated with insulin (16 units) and an ACE inhibitor, and has a mean HbA , of 6.4%.

The subject's mother (II-3) had a glycosuria complicated pregnancy when she was 33 years old. She was diagnosed to have Type 2 diabetes at the age of 38 years and was then treated with glibenclamide for 10 years. At the time of the study she had proliferative retinopathy, nephropathy, peripheral neuropathy, necrobiosis lipoidica, hypertension and cataracts. Insulin treatment (20 units) was commenced and her HbA_{1c} was reduced from 17.2% to 9.2% within 8 months. Two months later, she had a myocardial infarction followed by progressive deterioration of cardiac and renal functions. She died of pulmonary edema and septicaemia with a gangrenous foot at the age of 52 years.

The fourth daughter (III-6) had been treated with insulin since her incidental diagnosis of diabetes at the age of 12 years after a nasal polypectomy. She is currently receiving 68 units of insulin, and has mean HbA_{1c} of 8.8%.

Two other family members underwent screening by OGTT. The second daughter (III-3) has fluctuated between having normal glucose tolerance and IGT over the last 11 years. A brother (III-7) had overt diabetes on screening with an initial HbA_{1c} of 10.5%. Insulin was started after 3 months of dietary treatment. He is currently receiving 26 units of insulin, with a mean HbA_{1c} of 5.3%.

One maternal uncle (II-4) was diagnosed with diabetes and hyperlipidnemia with thirst and polyuria at the age of 39 years. He has been treated with oral drugs since diagnosis, and has mean HbA_{1c} of 8.4%. His children were not available for detailed genetic testing and clinical assessment. The affected members II-4, III-6 and III-7 (FIG. 10) have remained free of complications despite all having had diabetes for more than 10 years.

The father was also diagnosed with IGT. He was non-obese and had hyperlipidaemia.

Sequencing of the HNF-1_{α} gene in this family showed a novel splice acceptor site mutation (AG→AA) in intron 2 (IVS2nt-1G→A) which cosegregated with diabetes (FIG. 10) (Ng, et al (1999) Diabetic Medicine 16: 956). This mutation is expected to produce a nonfunctional mRNA. All the diabetic members, including the maternal uncle, (II4, III-2, III-5, III-6 and III-7) were heterozygous for this mutation but the father (II-2) and the daughter (III-3) with IGT did not have the mutation. Thus it is very likely that the mother (II-3) for whom no DNA sample was available also carried this mutation. As with other patients with HNF-1α diabetes (Byrne, et al (1996) Diabetes 45: 1503), most affected family members exhibited defective pancreatic beta-cell function as assessed by the glucagon stimulation test. The mother and all the affected siblings, except subject III-2, were insulin deficient based on a definition of post-glucagon (1 mg intravenously) stimulated plasma C peptide at 6 min of less than 0.6 nmol/1 (0.24-0.55 nmol/I respectively) (Service, et al (1997) Diabetes Care 20: 198). The brother, III-7, who was diagnosed with diabetes by OGTT was also insulin deficient. All the HNF-1α mutant carriers, except II-4, required insulin treatment for glycemic control.

Although all affected family members carried the same HNF-1α gene mutation, their clinical courses have varied tremendously. Severe complications were present in those family members whose diagnosis was delayed and who presumably had poor glycemic control before diagnosis (II-3, III-2 and III-5). Complications were, however, absent in the uncle (II-4) and the younger siblings (III-6 and III-7) despite now having had diabetes for more than 10 years (FIG. 10), who were promptly diagnosed and received treatment. This is in accordance with a recent report suggesting that poor glycemic control is associated with a twofold to threefold increased risk among MODY3 patients of developing microalbuminuria and retinopathy, respectively (Isomaa, et al (1998) Diabetologia 41: 467).

It is noteworthy that both maternal grandparents (I-3 and I-4) were diagnosed with diabetes diagnosed in their late 50 s. The effect of this bilineality on the natural course of HNF-1α diabetes in this family is uncertain. It is, however, possible the non-MODY maternal grandparent transmitted a modifier gene affecting the age at onset or severity of the diabetes in carriers with the HNF-1_{α} amutation. The age at diagnosis of diabetes in this family was increasingly younger with successive generations despite all carriers being relatively non-obese. This earlier diagnosis could be due to ascertainment bias or, more likely, an epiphenomenon due to increasing westernisation of the Hong Kong lifestyle with increased intake of high fat food and decreased physical activity (Chan and Cockram (1997) Diabetes Care 20:1785). This highlights the important influence of environment interacting with genetics in the natural course of HNF-1α diabetes. In conclusion, this report emphasizes the need for early diagnosis by glucose tolerance testing or genetic screening, and appropriate treatment in patients who have a strong family history of diabetes, especially those with early onset disease and insulin deficiency.

The data presented in Examples 1-5 demonstrate a combination of genetic mutations that are uniquely associated with the increased risk of a Chinese individual to develop type 2 diabetes. The mutations are exemplified by, but are not limited to G20R, A116V, IVS2nt→GA, R203H, S432C and I618M of HNF-1α; V101M, I110T, A119D, Q239R and G385V of glucokinase; S20G of amylin; and A3243G of mitochondrial tRNA^{Leu(UUR)}. Mutations correlative with a genetic predisposition of a Chinese individual to develop type 2 diabetes are efficiently identified in Chinese families with a positive family history of the disease, but find use in screening any Chinese individual that is asymptomatic but at risk of developing diabetes. Methods for identification of a combination of at least two genetic mutations correlative with type 2 diabetes in a Chinese individual offers an important tool for clinicians, not only to initiate prophylactic therapies before the onset of overt diabetic symptoms, but also to design therapies that are directed to the specific etiology of the disease in each individual.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporate by reference.

1. A microchip comprising:
a combination of at least two different mutant nucleic acid sequences of a wild-type nucleic acid sequence, wherein each wild-type nucleic acid sequence encodes a protein involved in insulin secretion, wherein said gene comprises at least one mutation indicative of a predisposition for type 2 diabetes in a member of a Chinese population.

2. The microchip according to claim 1, wherein said nucleic acid sequences comprise nucleic acid selected from the group consisting of genomic DNA, complementary DNA and messenger RNA.

3. The microchip according to claim 1, wherein said type 2 diabetes is maturity onset diabetes of the young.

4. The microchip according to claim 1, wherein said microchip further comprises a genetic marker that uniquely identifies a member of a Chinese population.

5. A microchip comprising:
a combination of at least two different nucleic acid sequences, wherein each nucleic acid sequence encodes a gene product involved in insulin secretion wherein said gene comprises at least one mutation indicative of a predisposition for type 2 diabetes in a human subject of a Chinese population, wherein said gene product is selected from the group consisting of a glucokinase, a hepatocyte nuclear factor 1α, an amylin and a mitochondrial tRNA (Leu) (UUR).

6. A microchip comprising:
at least one each of a combination of different nucleic acid sequences, wherein each nucleic acid sequence encodes a protein selected from the group consisting of glucokinase, hepatocyte nuclear factor la, amylin and mitochondrial tRNA(Leu)(UTR), wherein said glucokinase gene comprises at least one mutation selected from the group consisting of V101M, I110T, A119D, Q239R, and G385V, and said hepatocyte nuclear factor la gene comprises at least one mutation selected from the group consisting of G20R, A116V, IVS2nt-G→A, R203H, S432C, and I618M, and said amylin gene comprises the mutation S20G, and said mitochondrial tRNA(Leu)(UUR) gene comprises the mutation A3243G.

7. A microchip comprising at least one nucleic acid sequence selected from the group consisting of SEQ ID NO:2, SEQ ID NO:7 and SEQ ID NO:10.

8. A microassay system comprising a microchip according to claim 1.

9. A kit comprising a microchip according to claim 1.

10. A nucleic acid primer comprised of SEQ ID NO: 34.

11. A nucleic acid primer comprised of SEQ ID NO: 35.

12. A nucleic acid primer comprised of SEQ ID NO: 36.

13. A nucleic acid primer comprised of SEQ ID NO: 37.

14. A nucleic acid probe that specifically anneals to a nucleic acid encoding a mutant gene of a wild-type gene involved in insulin secretion, wherein said mutant gene comprises at least one mutation indicative of increased risk for type 2 diabetes in a human subject of a Chinese population, and wherein said nucleic acid probe does not bind to said wild-type gene.

15. An isolated nucleic acid encoding a mutant gene of a wild-type gene that encodes a protein involved in the secretion of insulin, wherein said mutant gene comprises at least one mutation associated with increased risk for type 2 diabetes in a subject of a Chinese population.

16. The isolated nucleic acid according to claim 15, wherein said mutation is a single nucleotide polymorphism.

17. The isolated nucleic acid according to claim 15, wherein said mutation is selected from the group consisting of a missense, a nonsense, an insertion and a deletion mutation.

18. The isolated nucleic acid according to claim 15, wherein said wild-type gene encodes hepatocyte nuclear factor la, and said mutation is A116V.

19. The isolated nucleic acid according to claim 15, wherein said wild-type gene encodes glucokinase, and said mutation is selected from the group consisting of V101M and Q239R.

20. An isolated nucleic acid encoding a mutant gene of a wild-type gene that encodes a protein involved in the secretion of insulin, wherein said mutant gene is selected from the group consisting of SEQ ID NO: 2, SEQ ID NO: 7 and SEQ ID NO: 10.

21. An isolated amino acid sequence encoded by a mutant gene of a wild-type gene encoding a protein involved in the secretion of insulin, wherein said mutant gene comprises at least on mutation associated with increased risk for type 2 diabetes in a member of a Chinese population.

22. An antibody that specifically binds a protein encoded by a mutant gene of a wild type gene encoding a protein involved in the secretion of insulin, wherein said mutant gene comprises at least on mutation associated with increased risk for type 2 diabetes in a member of a Chinese population, and wherein said antibody does not bind to a protein encoded by said wild-type gene.

23. A method of determining a genetic predisposition of a member of a Chinese population to develop type 2 diabetes, said method comprising the step of: contacting a sample comprising nucleic acid from said member with a combination of at least two nucleic acid sequences, wherein each nucleic acid sequence encodes a mutant gene of a wild-type gene encoding a protein involved in insulin secretion, wherein each mutant gene comprises at least one mutation indicative of a predisposition of a member of a Chinese population to develop type 2 diabetes, whereby identification of at least one of said mutations in said sample is indicative of a genetic predisposition for type 2 diabetes in said member of a Chinese population.

24. A method for detecting an increased risk of an individual of a Chinese population with decreased insulin secretory function to develop type 2 diabetes, said method comprising the step of:
contacting a sample comprising nucleic acid from said individual with a combination of at least two different nucleic acid sequences, wherein each nucleic acid sequence encodes a mutant gene of a wild-type gene encoding a protein involved in insulin secretion, wherein each mutant gene comprises at least one mutation indicative of a predisposition of a member of a Chinese population to develop type 2 diabetes, wherein identification of at least one of said mutations in said sample is indicative of an increased risk for type 2 diabetes in said individual of a Chinese population.

25. The method according to claim 23, wherein said combination of at least two different nucleic acid sequences are attached to a microchip.

26. The method according to claim 23, wherein said nucleic acid sample is obtained from bodily fluid or tissue.

27. The method according to claim 23, wherein said wild-type gene encodes a gene product selected from the group consisting of hepatocyte nuclear factor 1α, glucokinase, amylin and mitochondrial tRNA(Leu)(UUR).

28. A method of determining a genetic predisposition of a member of a Chinese population to develop type 2 diabetes, said method comprising the step of:
contacting a sample comprising nucleic acid from said member with a combination of at least two different nucleic acid sequences selected from the group consisting of G20R, A116V, IVS2nt-G→A, R203H, S432C, and I618M of hepatocyte nuclear factor 1α; V101M, I110T, A119D, Q239R, and G385V of glucokinase; S20G of amylin, and A3243G of mitochondrial tRNA(Leu)(UUR), wherein each nucleic acid sequence encodes a mutant gene of a wild-type gene encoding a protein involved in insulin secretion, wherein each mutant gene comprises at least one mutation indicative of a predisposition of a member of a Chinese population to develop type 2 diabetes, and wherein said identification of one of said mutations in said sample is indicative of a genetic predisposition for type 2 diabetes in said member of a Chinese population.

29. A method for detecting an increased risk of an individual of a Chinese population with decreased insulin secretory function to develop type 2 diabetes, said method comprising the step of:
contacting a sample from said individual with a combination of at least two different nucleic acid sequences selected from the group consisting of G20R, A116V, IVS2nt-G→A, R203H, S432C, and I618M of hepatocyte nuclear factor 1α;V101M, I110T, A119D, Q239R, and G385V of glucokinase; S20G of amylin, and A3243G of mitochondrial tRNA(Leu)(UUR), wherein each nucleic acid sequence encodes a mutant gene of a wild-type gene encoding a protein involved in insulin secretion, wherein each mutant gene comprises at least one mutation indicative of a predisposition of an individual of a Chinese population to develop type 2 diabetes, and wherein the identification of at least one of said mutations in said sample is indicative of an increased risk for type 2 diabetes in said individual of a Chinese population.

30. A method for screening for genetic mutations in an individual of a Chinese population diagnosed with type 2 diabetes, said method comprising the steps of:
contacting a sample from said individual with a combination of at least two different nucleic acid sequences, wherein each nucleic acid sequence encodes a mutant gene of a wild-type gene encoding a protein involved in insulin secretion, wherein each mutant gene comprises at least one mutation indicative of a predisposition of a member of a Chinese population to develop type 2 diabetes, and wherein identification of at least one of said mutations in said sample is indicative of an etiology of said type 2 diabetes in said individual of a Chinese population.

31. The method according to claim 30, wherein said individual has been diagnosed with maturity onset diabetes of the young.

32. The method according to claim 30, wherein said individual has at least one primary family member that has been diagnosed with maturity onset diabetes of the young.

33. The method according to claim 30, wherein said mutation is selected from the group consisting of a missense, a nonsense, an insertion and a deletion mutation.

34. A method for screening for genetic mutations indicative of increased risk of an individual of a Chinese population to develop type 2 diabetes, said method comprising the steps of:
contacting a sample from said individual with a combination of at least two different nucleic acid sequences selected from the group consisting of G20R, A116V, IVS2nt-G→A, R203H, S432C, and I618M of hepatocyte nuclear factor 1α;V101M, I110T, A119D, Q239R, and G385V of glucokinase; S20G of amylin, and A3243G of mitochondrial tRNA(Leu)(UTR), wherein each nucleic acid sequence encodes a mutant gene of a wild-type gene encoding a protein involved in insulin secretion, wherein each mutant gene comprises at least one mutation indicative of a predisposition of an individual of a Chinese population to develop type 2 diabetes.

35. A method for screening for a genetic predisposition to develop type 2 diabetes in an individual of a Chinese population having at least one primary family member that has been diagnosed with type 2 diabetes, said method comprising the steps of:
contacting a sample comprising nucleic acid from said individual with a combination of at least two different nucleic acid sequences, wherein each nucleic acid sequence encodes a mutant gene of a wild-type gene encoding a protein involved in insulin secretion, wherein each mutant gene comprises at least one mutation indicative of a predisposition of a member of a Chinese population to develop type 2 diabetes, and wherein identification of at least one of said mutations in said sample is indicative of a genetic predisposition to develop type 2 diabetes in said individual of a Chinese population.


SEQUENCE LISTING (37 sequences)

SEQ ID NO: 1 - DNA, 726 bp, Homo sapiens
  mutation (147): g to a mutation
tgccggccggcaggcaaacgcaacccacgcggtgggggaggcggctagcgtggtggacccgggccgcgtggccctgtggcagccgagccatggtttctaaactgagccagctgcagacggagctcctggcggccctgctcgagtcaaggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcagtaaggagccctgccccgtccccgctcccaggagagcctagaggggcccccctcagctcctaacgagccccccttctgagttgagtccccatgaccttcagcctttagcctagttgctgggaagggggacagggcccatgagagcccaggggtccttgcttggaggtttgagcctccagcccctgaactgctcctctgcagagtcccaaatcccatgagcccaggcctttagcccagtccttgggcnagggggacatttcccagggggtccaagatgggagaaaaagcagtgaattcacaactcaaatgcc

SEQ ID NO: 2 - DNA, 852 bp, Homo sapiens
  mutation (425): c to t mutation
cacccacccatccatccatccgtccatccacccattcatccattcatccattcacccatccatccatccacatatcttcatctgtgttgtgtgtctgtgtatccatgtttctaaacctttatctgttccagtgtctgtatccataggcctgtgtccacgtttgtcatgtgtgtgcgtcnacaagtctctgtcctcatgaccatgtgtctgtgtccctgtgtcctggcataaatgaccatacctcaccgtccctgagtctatgtgtaggcccctgggctccataactgctttcatgcacagtccccaccctcagagttgacaaggttccagcacccaggaccgcagccccacctatggggagagacagcccttgctgagcagatcccgtccttgccctctcccagggaggacccgtggcgtgtggtgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcgtaagtaatgaccctaccccgcatcttccctgggagggcccaggactctcccctaactcataggtgggggctggaagcttcaccatccccattacacagacaggtagatggaaaggaagtcagtgggattcaacctgcatttattacctattctgcgccaggcactctgtgggacgggagtanacttggtcctgaacatccaaagatgaatgaaatgggtccctgctttctttttctttttttagata

SEQ ID NO: 3 - DNA, 1381 bp, Homo sapiens
  mutation (383): g to a mutation
cgtgactctggaaaaatatgtaagctctctgagcctcagcttcttcatctgtacaatggggatagtaaatgtgccaaatcagaacaaatgctaatgcttacctgcagtcttgtactgagaaggatggtgagatcatatcttgggttggtaggaaagcattcagggattgattagtgatgtttgccttgaacacaggttaagaaagtgatggcatgtgtgctgtgtgtttgtcatcagtagattagatgatttctaagttctagctgtaagctcctctggttcagcgccatggcaatgagaaagaatcaagggcaaggtcaggggaatggacgagggaaggtgagagtggccagtaccccactcacggctttctgtgcctgcaaagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaataggtacaacggcgggcgggaaacagtgctggtttggtctgggctgcggcaaggccaggggaaggggaaggtgactctaggtcctgtaaaaggctgtccagttgccgagaactcctgatattggcttagcctggcccagaaaattgagaatacttgaacctaagcccattcctcgcagcccccctgcaccntggacaccaagcaaccccttccatggatgctcacccaattcgattctctctacaatcctatggctcttttgctcactttatgaatggagagactgaggtcagacagactgtcaattgcccaaggtcacacagcagacctggcattggaacccagatctgccagcctcaaaccctccggcagagntcagcttctcagaaccctccccttcatgcccaggacagggttcctctgagcctggcctggaggctcatgggtggctatttctgcagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtaagtggtatgtggggacaagggacacgtgggaaggtgggagggttggggaggactgtcccattgacagcagtcacctaaacctctttgcacgtcagtttggttccattc

SEQ ID NO: 4 - DNA, 1381 bp, Homo sapiens
  mutation (465): g to a mutation
cgtgactctggaaaaatatgtaagctctctgagcctcagcttcttcatctgtacaatggggatagtaaatgtgccaaatcagaacaaatgctaatgcttacctgcagtcttgtactgagaaggatggtgagatcatatcttgggttggtaggaaagcattcagggattgattagtgatgtttgccttgaacacaggttaagaaagtgatggcatgtgtgctgtgtgtttgtcatcagtagattagatgatttctaagttctagctgtaagctcctctggttcagcgccatggcaatgagaaagaatcaagggcaaggtcaggggaatggacgagggaaggtgagagtggccagtaccccactcacggctttctgtgcctgcagagttcacccatgcagggcagggagggctgattgaagagcccacaggtgatgagctaccaaccaagaaggggcggaggaaccatttcaagtggggcccagcatcccagcagatcctgttccaggcctatgagaggcagaagaaccctagcaaggaggagcgagagacgctagtggaggagtgcaataggtacaacggcgggcgggaaacagtgctggtttggtctgggctgcggcaaggccaggggaaggggaaggtgactctaggtcctgtaaaaggctgtccagttgccgagaactcctgatattggcttagcctggcccagaaaattgagaatacttgaacctaagcccattcctcgcagcccccctgcaccntggacaccaagcaaccccttccatggatgctcacccaattcgattctctctacaatcctatggctcttttgctcactttatgaatggagagactgaggtcagacagactgtcaattgcccaaggtcacacagcagacctggcattggaacccagatctgccagcctcaaaccctccggcagagntcagcttctcagaaccctccccttcatgcccaggacagggttcctctgagcctggcctggaggctcatgggtggctatttctgcagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtaagtggtatgtggggacaagggacacgtgggaaggtgggagggttggggaggactgtcccattgacagcagtcacctaaacctctttgcacgtcagtttggttccattc

SEQ ID NO: 5 - DNA, 774 bp, Homo sapiens
  mutation (642): c to g mutation
gcagctgacccagggattggcaaaaggtagaaacaaaggcagatttgctggctgcataaaggcagacaggcagatggcctaagcaaaccaatggagtttgaagtgctgagggctgtggaggcaggggagggcagggaagtggggtgctgaggcaggacactgcttccctctccaggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtgagtgtccttgcttgtaaggaaaacccaacctcatctttccttggcagggagattctggagcagtccctagggaggccctgtggggaccccggccccccggacacagcttggcttcccctcgtaggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctgcaccctggtcatcggtaagctggtggggatgggtgggcacctgggtgggaggctcatggggcaaccgcanaatccaggagctggaaaagccactgggactcattcattcattcattcattcatacaacatgt

SEQ ID NO: 6 - DNA, 407 bp, Homo sapiens
  mutation (320): c to g mutation
tccagtgttcacagtaagatgtactcaggccagtccatgggcggccgtggaccctggctgggaggctccctttgttaagaaccgagggtagaggtgtgactttggggttcctgttatgtgctgtgatccaggaggtgtggccctgcctccccatcctgagtacccctagggacaggcaggtggggtgggtgtgggtgcctggtgggtggctagcagccttgtttgcctctgcagtgtcctccagcagcctggtgctgtaccagagctcagactccagcaatggccagagccacctgctgccatccaaccacagcgtcatggagaccttcatctccacccagatggcctcttcctcccagtaaccacggcacctgggccctggggcctgtactgcctgcttggggggt

SEQ ID NO: 7 - DNA, 434 bp, Homo sapiens
  mutation (212): g to a mutation
tcccttgtgccttccctcctcctctttgtaatatccggctcagtcacctggggcccacccagcccaaggccagcctgtgggtgtccctgaggctgacacacttctctctgtgcctttagaagtcggggacttcctctccctggacctgggtggcactaacttcagggtgatgctggtgaaggtgggagaaggtgaggaggggcagtggagcatgaagaccaaacaccagatgtactccatccccgaggacgccatgaccggcactgctgagatggtgagcagcgcaggggccggggcagggggcaaggcatgcaggatctcagggcccagctagtcctgacgggaggtgccacctgtctaccaggggtggggagagcgggggctggaggaccacccagcctcagaggcagctggaggcctgggtgaacaggact

SEQ ID NO: 8 - DNA, 434 bp, Homo sapiens
  mutation (240): t to c mutation
tcccttgtgccttccctcctcctctttgtaatatccggctcagtcacctggggcccacccagcccaaggccagcctgtgggtgtccctgaggctgacacacttctctctgtgcctttagaagtcggggacttcctctccctggacctgggtggcactaacttcagggtgatgctggtgaaggtgggagaaggtgaggaggggcagtggagcgtgaagaccaaacaccagatgtactccacccccgaggacgccatgaccggcactgctgagatggtgagcagcgcaggggccggggcagggggcaaggcatgcaggatctcagggcccagctagtcctgacgggaggtgccacctgtctaccaggggtggggagagcgggggctggaggaccacccagcctcagaggcagctggaggcctgggtgaacaggact

SEQ ID NO: 9 - DNA, 434 bp, Homo sapiens
  mutation (267): c to a mutation
tcccttgtgccttccctcctcctctttgtaatatccggctcagtcacctggggcccacccagcccaaggccagcctgtgggtgtccctgaggctgacacacttctctctgtgcctttagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgatgagatggtgagcagcgcaggggccggggcagggggcaaggcatgcaggatctcagggcccagctagtcctgacgggaggtgccacctgtctaccaggggtggggagagcgggggctggaggaccacccagcctcagaggcagctggaggcctgggtgaacaggact

SEQ ID NO: 10 - DNA, 492 bp, Homo sapiens
  mutation (105): a to g mutation
ggcaggaaccaggccctactccggggcagtgcagctctcgctgacagtccccccgacctccaccccaggcacgggctgcaatgcctgctacatggaggagatgcggaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtaaggatgcccccctcccccacaacccaggccctgggcgctctggtgcagcggcagatgggagccgggccattgcagataatgggcttgtttttaaacaactctggggaaaagcaaactgacaatccgttcgtaagctccatcccttctgctcagtcatgacctgcccctgtgagagatgaagggttagtcccagttgtgatgtgataagcccagacctctttccttccgacaggtgat

SEQ ID NO: 11 - DNA, 532 bp, Homo sapiens
  mutation (248): g to t mutation
gctgggggacggctggccggggcccctccctggagaacgagaggccgccgctggagggggatggactgtcggagcgacactcagcgaccgccctacctcctcccgccccgcagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggtgctggcgggcgtcatcaaccgcatgcgcgagagccgcagcgaggacgtaatgcgcatcactgtgggcgtggatggctccgtgtacaagctgcaccccaggtgagcctgccccgctctctccctggtaaagtggggcccaaaaagcgcgcgctccaaggttccttgcggttcccaagctccaagatttcgtagtcctcttctcgtcccccttggcctagatttgggggaagggtcgactgcgtgcagggcgcccggtaatgaatgtggaggatgaggtgggagga

SEQ ID NO: 12 - DNA, 1481 bp, Homo sapiens
  mutation (254): a to g mutation
tgtcaaaaaatctcagccatctagggtgtttgcaaccaaacactgagttacttatgtgaaaaattgttttccttttggggtttttcaatccaattacaagaatatttgatgtcacatggctggatccagctaaaattctaaggctctaacttttcacactttgttccatgttaccagtcatcaggtggaaaagcggaaatgcaacactgccacatgtgcaacgcagcgcctggcaaattttttagttcattccggcaacaactttggtgccattctctcatctaccaacgtgggatccaatacatatggcaagaggaatgcagtagaggttttaaagagagagccactgaattacttgcccctttagaggacaatgtaactctatagttattgttttatgttctagtgatttcctgtataatttaacagtgcccttttcatctccagtgtgaatatatggtctgtgtgtctgatgtttgttgctaggacatataccttctcaaaagattgttttatatgtagtactaactaaggtcccataataaaaagatagtatcttttaaaatgaaatgtttttgctatagatttgtattttaaaacataagaacgtcattttgggacctatatctcagtggcacaggtttaagaacgaaggagaaaaaggtagtttgaaccttggtaaattgtaaacagctaataatgaagttattcttgacatgagaaaatcagtaattggaccaggcgcggtggctcttgcctgtaatcccagcactttgggaggccgaggcaggcagatcacaaggtcaggagttcgagaccagcctgaccaacatggtgaaaccctgtctctactaaaaatacaaaaattagccgggggtggtgacatgtgcctgtaatcccagctactcaggaggctaaggcaggagaatcgcttaaacccaggaggcggaggttgcagtgagccgagattgcaccactgcactccagcctgggtggcagagtgagactcgtctcaaaaaaaagaaagaaaattagtaattgtaagtacccctgataagcaaattagtaattgtcaatacccctgttaagcaattcctttttgcagtatatttctgaaatgacagaatgctgttttaaaaacaaagaaataaaatcctgctcctgactcggtcaaaatattttttaaagtctattgtttgttgtgcttgctggtactaagaggctatttaaaagtataaaactgctttgtatccatgagggtttcattgtgtgttagcagcagtgagcttctattaaatgtatatgtcatttattttgtttaagtggctttcagcaaacctcagtcatattcttatgcagggtattgcgaaacaacttgtgttctattaatcgtgtcttcaattaaaagaccacagacttctggaaactctttgctgtataaagattattctttgttaacaaattagacattctagcaaagt

SEQ ID NO: 13 - DNA, 480 bp, Homo sapiens
  mutation (243): a to g mutation
ggacatcccgatggtgcagccgctattaaaggttcgtttgttcaacgattaaagtcctacgtgatctgagttcagaccggagtaatccaggtcggtttctatctaccttcaaattcctccctgtacgaaaggacaagagaaataaggcctacttcacaaagcgccttcccccgtaaatgatatcatctcaacttagtattatacccacacccacccaagaacagggtttgttaagatggcagggcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaa

SEQ ID NO: 14 - DNA, 21 bp, Homo sapiens
ggcaggcaaacgcaacccacg

SEQ ID NO: 15 - DNA, 20 bp, Homo sapiens
cagtgcctctttgctcaggc

SEQ ID NO: 16 - DNA, 21 bp, Homo sapiens
tgcctgcagagttcacccatg

SEQ ID NO: 17 - DNA, 30 bp, Homo sapiens
atctgctgggatgctgggccccacttgcaa

SEQ ID NO: 18 - DNA, 21 bp, Homo sapiens
tggagcagtccctagggaggc

SEQ ID NO: 19 - DNA, 21 bp, Homo sapiens
gttgccccatgagcctcccac

SEQ ID NO: 20 - DNA, 21 bp, Homo sapiens
gtacccctagggacaggcagg

SEQ ID NO: 21 - DNA, 21 bp, Homo sapiens
accccccaagcaggcagtaca

SEQ ID NO: 22 - DNA, 21 bp, Homo sapiens
gggcaaggtcaggggaatgga

SEQ ID NO: 23 - DNA, 21 bp, Homo sapiens
cagcccagaccaaaccagcac

SEQ ID NO: 24 - DNA, 21 bp, Homo sapiens
gtccctgaggctgacacactt

SEQ ID NO: 25 - DNA, 22 bp, Homo sapiens
agctgggccctgagatcctgca

SEQ ID NO: 26 - DNA, 21 bp, Homo sapiens
acctgggtggcactaacttca

SEQ ID NO: 27 - DNA, 26 bp, Homo sapiens
cggcccctgcgctgctcaccatctga

SEQ ID NO: 28 - DNA, 21 bp, Homo sapiens
ggactgtcggagcgacactca

SEQ ID NO: 29 - DNA, 21 bp, Homo sapiens
gcggttgatgacgcctgccag

SEQ ID NO: 30 - DNA, 20 bp, Homo sapiens
tcacatttgttccatgttac

SEQ ID NO: 31 - DNA, 22 bp, Homo sapiens
caataactatagagttacattg

SEQ ID NO: 32 - DNA, 20 bp, Homo sapiens
aaggttcgtttgttcaacga

SEQ ID NO: 33 - DNA, 20 bp, Homo sapiens
agcgaagggttgtagtagcc

SEQ ID NO: 34 - DNA, 21 bp, Homo sapiens
catgcacagtccccaccctca

SEQ ID NO: 35 - DNA, 19 bp, Homo sapiens
tcccactgacttcctttcc

SEQ ID NO: 36 - DNA, 20 bp, Homo sapiens
aggaaccaggccctactccg

SEQ ID NO: 37 - DNA, 21 bp, Homo sapiens
tactccagcaggaactcgtcc